(19) United States
(12) Patent Application Publication
(10) Pub. No.: US 2012/0226271 A1
(43) Pub. Date: Sep. 6, 2012

(54) Vacuum Ablation Apparatus and Method

(76) Inventors: Peter Callas, Castro Valley, CA (US); Robert M. Pearson, San Jose, CA (US)

(21) Appl. No.: 13/472,557

(22) Filed: May 16, 2012

Related U.S. Application Data
(63) Continuation-in-part of application No. 13/080,437, filed on Apr. 5, 2011.
Application No. 13/080,437 is a continuation of application No. 11/388,724, filed on Mar. 24, 2006, now Pat. No. 7,942,873.
(60) Provisional application No. 60/665,407, filed on Mar. 25, 2005.

(51) Int. Cl. A61B 18/18; A61B 18/14

(52) U.S. Cl. 606/33; 606/41

(57) ABSTRACT

Ablation devices and associated methods are provided for use in ablating a target tissue, such as a cystic lesion. The ablation apparatus includes an integral or connected elongate probe and a deployable structure that is axially slidable along the outer surface of the electrode. The electrode can be disposed at the probe's distal end region for ablating tissue when the electrode(s) are activated to create an ablated margin of tissue at least partially surrounding the target tissue. Suction can be applied with a vacuum source operably connected to the proximal end region of the deployable structure. A target tissue, such as a cystic lesion, can be drawn against the surface of the electrical probe.

### CROSS-REFERENCE TO RELATED APPLICATIONS

This application is a continuation in-part application U.S. application Ser. No. 13/080,437, which application is a continuation of U.S. application Ser. No. 11/388,724, filed Mar. 24, 2006, which claims priority to U.S. Provisional Application No. 60/665,407, filed Mar. 25, 2005, both of which are incorporated in their entireties herein by reference. This application is also related to U.S. non-provisional application Ser. Nos. 12/488,070, filed Jun. 19, 2009; 12/751,826, filed Mar. 31, 2010; and 12/751,854, filed Mar. 31, 2010; all of which are incorporated by reference herein.

### BACKGROUND

According to the American Cancer Society (ACS), about 9,420 new soft tissue cancers would be diagnosed in the United States in 2005. During 2005, 3,490 to Americans are-expected to die of soft tissue cancers. The five-year survival rate for people with soft tissue sarcomas is around 90% if the cancer is found while it is small and before it has spread. In contrast, the five year survival rate is between 10% and 15% for sarcomas that have metastasized (www.cancer.org).

Surgery is the oldest form of treatment for cancer. Advances in surgical techniques have allowed surgeons to successfully operate on a growing number of patients. Today, less invasive operations are often done to remove tumors while preserving as much normal function as possible.

Complete local excision is generally considered adequate treatment for benign soft tissue tumors. Treatment of localized primary and recurrent sarcomas, however, may involve various treatment approaches, including surgery alone or surgery combined with radiation therapy or chemotherapy. With this method, the entire lesion is surgically removed. Many sarcomas appear to be well demarcated grossly. However, microscopically, there is usually a pseudocapsule with foci of infiltrating tumor. Removal of the tumor along this apparent plane may leave gross or microscopic sarcoma behind. Additionally, as many as 27% of patients develop local recurrence or distant metastases following surgical resection in addition to adjuvant therapy (www.emedicine.com).

Excisional biopsy may further be safely performed for small superficial tumors (approximately <5 cm in diameter) or those known to be benign.

According to the ACS, breast cancer is the most common cancer among women excluding non-melanoma skin cancers. In 2002, the American Cancer Society estimated there were 203,500 new invasive and 54,300 new cases of in situ breast cancer among U.S. women, resulting in the deaths of almost 40,000 women, ranking second among cancer deaths in women, behind lung cancer.

Over a lifetime, one in seven American women will experience breast cancer. Surgery, in one form or another, is still the primary approach to reduction or elimination of tumor mass in the breast. With earlier detection making it possible for breast cancer to be diagnosed while it is still localized (in situ), surgery (especially minimally invasive, breast conserving surgery) is increasingly a more effective tool in the treatment of this form of cancer.

It has been suggested to ablate a margin of a lumpectomy cavity with a cryogenic or radiofrequency device (Klimberg et al., U.S. Appl. 2005/0000525A1). The radiofrequency device is placed in the cavity and purse-string sutures are used to pull the tissue surrounding the device together. Electrodes are deployed from the distal end of the device and activated. However, the surgeon must estimate the position of the electrodes in the cavity to ablate the margins of the cavity. Further, the method is complicated as the surgeon must place the sutures and then the device must be held in place while the sutures are closed.

### SUMMARY

In one aspect, the invention provides an apparatus for use in for ablating the margins of a cavity such as a surgical cavity formed in a target tissue or on the surface of a target tissue. In one embodiment, the apparatus includes an ablation device having an elongate probe having distal and proximal end regions and one or more electrodes disposed at the probe's distal end for ablating tissue when radiofrequency energy, microwave energy, or electrical pulses for reversible or irreversible electroporation (IRE), is applied to the electrodes. The apparatus includes at least one opening in the distal end region of the probe at which suction can be applied to the proximal end region of the apparatus to allow ablation of tissue to be drawn against the apparatus. Preferably, the one or more electrodes are aligned with the one or more openings, to allow deployment of the electrodes through the openings and ablation of tissue drawn against the openings when a vacuum is applied to the sleeve.

The apparatus may also include an insulating thermal barrier positioned around at least a portion of the distal end of the probe. The thermal barrier is preferably formed of a low conductivity material. The apparatus may further include a sealing plate disposed at the proximal end region of the probe that is adapted to be pressed against a patient's surgical site, when the apparatus is inserted into the surgical cavity formed in the patent, to cover and seal the opening of the cavity.

In one embodiment, the apparatus further comprises at least one temperature sensor positioned at least one of (i) on the sealing plate for measuring the temperature at the surface of the surgical cavity, and (ii) on the sleeve for sensing temperature within the surgical cavity. In another embodiment, the apparatus includes at least one temperature sensor positioned on one or more of the thermal barrier surfaces. At least one thermal sensor may further be positioned between the thermal barrier and the sealing plate. At least one of the electrodes may also include a thermal sensor. It will be appreciated that all or some of the electrodes may include a thermal sensor. In a particular embodiment, each of alternating electrodes includes a thermal sensor.

The distal end of the probe may include a chamber that communicates with the openings and the proximal end region where the vacuum is applied. The apparatus may also include at least one vent positioned in the proximal portion of the probe that communicates with the distal end portion to provide air flow through the probe. Further, the apparatus may include a covering positioned around at least a portion of the distal-end of the probe and covering at least a portion of the opening.

In another aspect, the invention provides a method for ablating margins of a cavity such as a surgical cavity formed in a tissue or ablating margins of a target tissue. The method includes (a) inserting an elongate probe into the cavity or the target tissue, (b) applying suction at surface regions of the probe within the cavity or the target tissue, thereby to draw wall portions of the tissue into contact with the probe surface regions, wherein tissue margins in the surgical cavity or the target tissue surround at least a portion of the probe, and (c) while maintaining suction at the surface regions, ablating the tissue margins.

In one embodiment, step (c) includes (ci) introducing one or more electrodes into the tissue margins, and (cii) applying radiofrequency energy, microwave power, or electrical pulses for reversible or irreversible electroporation of the target tissue to the electrodes until the margins have been ablated. In another embodiment, step (ci) includes deploying a plurality of electrodes into the margins of a target tissue at radially spaced intervals that, with the application of radiofrequency or other types of power to the electrodes, such as those disclosed herein, in step (cii) define an ablation volume surrounding the probe and including the margins.

In one embodiment, the probe includes a plurality of radially spaced openings through which suction is applied to the surface region, and the electrodes are deployed through the openings in step (ci). In another embodiment, air flow is provided between the cavity through the distal end of the probe to and from a vent positioned in the probe. In another embodiment, after ablation of at least a portion of the cavity or the surface of the target tissue, suction is discontinued, the probe is repositioned within the cavity or the target tissue, and the method repeated.

In yet another aspect, the invention provides an adapter for use with an ablation device of the type having (i) an elongate probe having distal and proximal end regions and (ii) one or more electrodes disposed at the probe's distal end region, for ablating tissue when power (such as electrical, radiofrequency, or microwave power) is applied to the electrode(s). The adapter includes an elongate sleeve having distal and proximal end regions and is adapted to be placed over the distal end region of the probe. In one embodiment, the adapter includes a plurality of openings in the sleeve distal end region (i) at which a suction can be applied with a vacuum source operably connected to the proximal end region of the sleeve, and (ii) which are alignable with the one or more electrodes, to allow ablation of tissue drawn against the openings when the suction is applied to the sleeve, by application of power applied to the electrode(s).

In one embodiment, the adapter includes a thermally insulative barrier positioned around a distal portion of the sleeve. In a further embodiment, the adapter further includes a sealing plate disposed on the sleeve's distal end region that is adapted to be pressed against a target tissue or a patient's surgical site, when the probe is inserted into the surgical cavity formed in the patent, to cover and seal the opening of said cavity. The sealing plate may be axially slidable along the proximal end region of the sleeve.

In one embodiment, the sealing plate is configured to fit over a portion of a female patient's breast. The adapter may further include means on the sleeve for limiting the axial movement of the sealing plate toward the sleeve's distal end. In another embodiment, the sleeve further includes at least one marker indicating the position of the sealing plate relative to the distal end of the probe.

In another embodiment, the adapter also includes an indicator on the sealing plate that indicates a sensed patient temperature. In one embodiment, the sealing plate includes at least one temperature sensor operatively connected to the indicator for sensing temperature at the surface of the surgical cavity. In another embodiment, the adapter further includes at least one temperature sensor on the sleeve and operatively connected to the indicator for sensing temperature within the surgical cavity. In yet another embodiment, at least one temperature sensor is positioned on at least one surface of the thermal barrier.

The adapter may include a multi-position lock at the sleeve proximal region for locking the position of the probe within the sleeve. In another embodiment, the adapter may include a lateral slide for aligning the probe within the sleeve.

The sleeve openings may have a microporous surface. In another embodiment, the adapter includes a semi-porous or porous sheath positioned over at least a portion of the openings.

In one embodiment, the sleeve, when placed over the probe's distal end region, forms a chamber therewith that communicates with the openings and with a port at the proximal region of the sleeve.

The adapter may also include an overflow relief valve positioned on the sealing plate.

The adapter may further include a valve and connection to the distal end portion of the sleeve to allow air flow through the adapter.

A method for electrically ablating a target tissue is presented. The method involves positioning a probe assembly in or near the target tissue to be ablated, wherein the probe assembly comprises a probe having a proximal end, a distal end, and an outer surface; and a deployable structure having an inner surface. At least a portion of the deployable structure surrounds at least a portion of the outer surface of the probe. The method also includes positioning the deployable structure near the target tissue; applying suction to at least a portion of the probe assembly; and applying electrical energy to the probe to electrically ablate the tissue.

Another method for electrically ablating a target tissue is presented herein. This method includes positioning a probe in or near a tissue to be ablated, wherein the probe comprises a proximal end and a distal end, wherein at least one opening is positioned at the distal end of the probe, and wherein at least one electrode is aligned with the at least one opening; applying suction at the at least one opening of the positioned probe to draw at least a portion of the tissue to be ablated into contact with the at least one opening of the probe; and applying electrical energy through the at least one electrode to electrically ablate the tissue.

A probe assembly is presented herein. The probe has a proximal end, a distal end, an outer surface, and a tissue contacting surface, wherein the tissue contacting surface comprises a deployable structure, wherein at least a portion of the deployable structure surrounds at least a portion of the outer surface of the probe, and wherein the deployable structure is configured to be fixed to the outer surface of the probe and the tissue.

In another embodiment, a shield having a lumen can surround an electrode and can be axially slidable along the electrode. Vacuum suction can be applied through the lumen of the shield to adhere the shield to the surface of a target tissue on a patient's skin.

These and other features of the invention will become more fully apparent when the following detailed description is read in conjunction with the accompanying drawings.

### BRIEF DESCRIPTION OF THE DRAWINGS

FIG. 1A is an illustration of an embodiment of the device for ablating the margins of a surgical cavity;

FIG. 1B is an illustration of another embodiment of the device for ablating the margins of a surgical cavity;

FIG. 2A is an illustration of the device of FIG. 1B showing the sleeve detached from the probe;

FIG. 2B is an illustration of the device of FIG. 1A showing a modular aspect with the sleeve detached from the probe;

FIG. 3 is a scanned image of an embodiment of a suction ablation device;

FIG. 4 is a detailed view of the apparatus distal portion of the device of FIG. 1A with a detailed view of the sleeve distal area;

FIG. 5 is an illustration of a detailed view of a locking mechanism;

FIGS. 6A-6C are illustrations of the positioning of the probe within the sleeve;

FIGS. 6A-6B show the probe in alternative positions using the locking mechanism of FIG. 5, FIG. 6C shows a linear slide mechanism;

FIG. 7 is a scanned image of an embodiment of the device for ablating the margins of a surgical cavity;

FIGS. 8A-8D show using the device for ablation of a lumpectomy cavity in a breast; and

FIG. 9 is an illustration of the positioning of the probe showing locations of the thermal sensors;

FIGS. 10A and 10B illustrate an ablation device having an axially slidable deployable structure for suction of tissue;

FIGS. 10C AND 10D illustrate cross-sections along lines C-C and D-D of FIGS. 10A and 10B, respectively.

FIGS. 10E and 10F illustrate cross-sections along lines C-C and D-D of FIGS. 10A and 10B, respectively, of an alternative embodiment of the device illustrated in FIGS. 10A and 10B.

FIGS. 11A and 11B illustrate alternative embodiments of the distal end of the device of FIGS. 10A and 10B.

### DETAILED DESCRIPTION

### I. Definitions

The terms below have the following meanings unless indicated otherwise.

“Radio Frequency” or “RF” refers to an electrical current that alternates the poles in the radio frequency range (extending from below 3 kHz to about 300 gigahertz).

“Soft tissue” refers to non-bone tissue.

A “tumor” or “lesion” refers to an abnormal lump or mass of tissue. Tumors or lesions can be benign (not cancerous) or malignant (cancerous).

“Cancer” as used herein refers to all types of cancer regardless of subset, therefore encompassing sarcoma, carcinoma, and other forms of cancer, invasive or in situ.

“Resection” refers to surgery to remove part or all of an organ or other structure.

“Distal end” with respect to an ablating instrument or introducer thereof, refers to the distal end or distal end region of the instrument, probe, or introducer thereof.

“Distal-end structure” or “distal-end member” refers to the ablating structure, e.g., needle, antenna, or electrode, carried at or deployable from the distal end of an ablating instrument or introducer thereof.

“Activating” or “activation”, in the context of activating a distal end structure, e.g., electrode, refers to the application of a stimulus to the structure that is effective to ablate tumor tissue in contact with the structure. Such activation can include electrical pulses for reversible or irreversible electroporation, RF energy, or microwave current applied to an electrode, current applied to a resistive heating element, ultrasound-generating current applied to an ultrasound generator or sonicator tip, a cryogenic fluid circulated through a circulation pathway in the probe, or an ablative fluid, e.g., ethanol or high salt, or any other desired fluid, such as, but not limited to saline or D5W ejected from the end of a needle.

The term “vacuum” as used herein refers to a space at least partially exhausted of air using a vacuum source such as an air pump. Specifically, the term refers to a degree of rarefaction below atmospheric pressure.

“Suction” as used herein refers to reducing the air pressure using a source of suction such as an air or vacuum pump.

Disclosed herein are devices and methods for performing vacuum-assisted ablation. In particular, the methods also involve using a medical device to deliver electrical pulses to a target tissue within a non-thermal irreversible electroporation range. A probe comprising at least one electrode is adapted to receive from a voltage generator a plurality of electrical pulses in an amount sufficient to cause non-thermal destruction of the target tissue. The number of pulses, pulse length, and pulse amplitude can be used to irreversibly electroporate the target tissue.

Throughout the present teachings, any and all of the one, two, or more features and/or components disclosed or suggested herein, explicitly or implicitly, may be practiced and/or implemented in any combinations of two, three, or more thereof, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the claimed inventions presented herein or in any future applications claiming priority to the instant application.

### II. Apparatus

The cavity ablation system of the invention generally includes an instrument or device for use in ablating the margins of a cavity such as a surgical cavity or a target tissue. Resection of tumors may be performed in open surgery or percutaneously. In open resection, the surgeon typically makes an incision in the skin and excises the tumor and a margin of healthy tissue surrounding the tumor. The pathology of the excised tissue is reviewed using standard cytological techniques and the margin is determined to be negative, close or positive. Typically, a second surgery is required for close or negative margins. In the United States, nearly 40% of patients require a second surgery for close or positive margins on resection (Henry-Tillman, et al., Semin. Surg. Oncol., 20(3):206-213, 2001). The goal of the resection is to obtain a negative margin, where no tumor cells are found, preferably within at least 1 cm of the edge of the resection. It will be appreciated that the device may further be used in any body cavity or on any target tissue surface where ablation of the tissue surrounding the cavity is necessitated.

In one aspect, the present device provides for ablation of the tumor bed after excision to provide an ablated margin surrounding the tumor bed. Margins of 0.5 to 3.5 cm, inclusive, can be ablated around the tumor bed. In one exemplary embodiment, a margin of at least about 1-2.5 cm is ablated at least partially surrounding the tumor bed. This ablated margin reduces the need for further surgery for resections with close or even positive margins. The ablated margin may further reduce the recurrence of tumor in the bed by providing an ablated margin at least partially surrounding the tumor bed even where the cytology results in a negative margin.

Generally, the devices and methods described herein are suitable for use in ablating soft tissue tumor beds (a.k.a. surgical cavities) such as those resulting from breast lumpectomies, removal of tumors in the brain, or other surgical procedures in which a cavity is created. In other embodiments, the devices and methods described herein can be suitable for use in ablating the surface of a target tissue, removing fluid from a cyst, or collapsing an aneurysm. Depending upon the procedure, approximately 5 mm to 2 cm of tissue may be removed; however, the amount of tissue may be more or less depending on the size of the tumor, the procedure used for resection, and the physician, among others. It will be appreciated that the device may be sized in accordance with the size of the target tissue or the body cavity. For example, the distal end of the device may be adjusted in length to accommodate the depth of the cavity. Further, multiple ablations may be used to ensure ablation of the cavity margins of various depth and/or width. It will further be appreciated the devices may be used in a body cavity or on the surface of a target tissue.

In one general embodiment, the devices described herein are placed at least partially in a tumor bed or other surgical cavity or tissue surface. Once positioned at a target tissue site in the cavity or within a target tissue, the apparatus can be configured to ablate tissue at that site as well as to create an ablated margin of tissue around the apparatus. The apparatus is formed of a probe or other elongate accessing member having a distal-end which is placed in the surgical cavity or into the target tissue. The distal end of the probe includes a series of tubular sections operatively connected to a suction source surrounding at least a portion of the probe distal end. The suction sections include at least one opening on the outside, that is, the side facing the cavity wall. In one exemplary embodiment, the at least one opening comprises a plurality of openings. The sections are connected at the proximal end to a source of suction, whereby when suction is applied, the target tissue or the tissue of the cavity wall is drawn adjacent the distal area of the probe. In this manner, the surgical cavity or any air gaps in the target tissue are “closed” against the probe. The probe further includes at least one activatable distal end region. In one exemplary embodiment, the distal end regions comprise one or more deployable electrodes or other activatable wires, antennas, or needles that can be deployed from the probe between the sections. The electrodes, when deployed, typically have a selected geometric configuration, such as a planar, or volume-forming configuration designed to ablate tumor tissue when activated. In another embodiment, the activatable distal end regions comprise one or more surface electrodes. In yet another embodiment, at least a portion of the probe distal end is activatable. The apparatus may further include a sealing member or plate disposed proximal to the distal region of the probe. As further discussed below with respect to the adapter, the plate is adapted to be pressed against a patient's surgical site to cover and preferably seal the opening of the cavity or the surface of the target tissue, allowing for a more efficient vacuum to be applied to the surgical cavity or the target tissue in order to draw at least a portion of the tissue to the probe and collapse the open spaces within the cavity and air gaps within the target tissue, whereby the peripheral tissue is brought into contact with the electrodes when deployed. The plate may be formed of a transparent material to allow for visual inspection of the cavity surface or the tissue surface. In another embodiment, the apparatus may include a thermally insulative barrier or skirt surrounding a portion of the distal end of the probe. This barrier serves to limit the thermal effect of the activatable regions to the area distal to the barrier, thus to define the ablation area and prevent burns to the skin. The barrier may be fixed to the distal end of the probe or be slidably attached to the probe. Where the device includes a sleeve, discussed further below, the barrier may be fixed to the sleeve or movably positioned around the sleeve. The apparatus may also include an intake or vent in the probe that communicates with the distal end of the probe to allow for at least a small amount of air flow to and from the cavity. In this manner, residual steam may be carried away from the ablation area.

In another embodiment, the apparatus makes use of commercially available ablation devices. In this embodiment, the device generally includes an elongate probe and a tubular sleeve, where the probe is positioned at least partially in the sleeve. The probe includes at least one distal-end structure adapted to be inserted into the walls of the cavity or into the target tissue, where the structure is activatable to ablate the walls of the cavity or the target tissue and create an ablated margin of tissue surrounding the cavity. It will be appreciated that the device includes connecting structures for connecting the distal-end structure to an activating device. The assembly, and particularly the suction instrument, of the invention will now be described with reference to the figures.

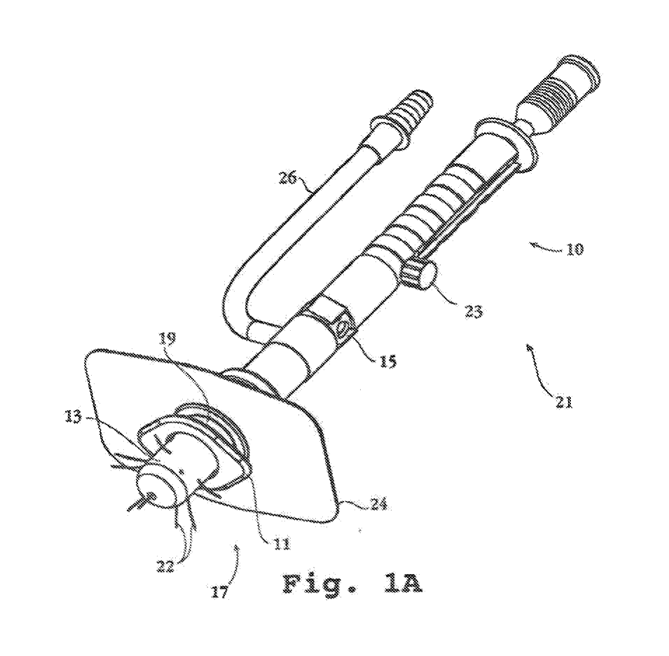

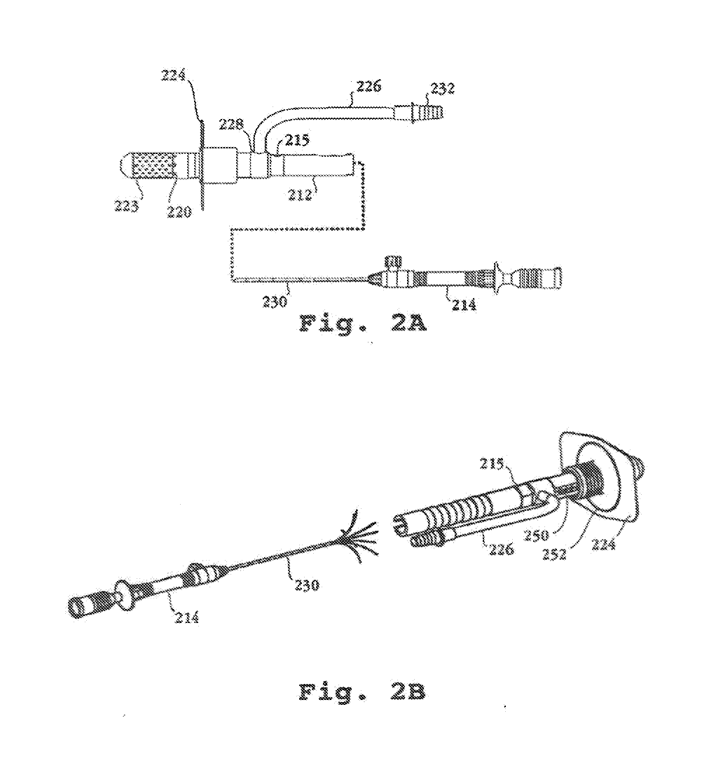

For convenience, similar element numbering is retained in FIGS. 1-11B to identify like structural features. For example, the sealing plate is numbered 24 in FIG. 1A, 224 in FIG. 2A, 424 in FIG. 4, etc.

In the embodiment seen in FIG. 1B, the apparatus 110 includes an ablation device 114 comprising an elongate probe having a distal region and a proximal region. In one embodiment, the probe includes at least one or more electrodes or antennas 122 deployable from the distal end of the probe for ablating tissue when activated. In one exemplary embodiment, a plurality of electrodes is deployable from the probe distal end. It will be appreciated the electrodes may be deployed radially or asymmetrically from the probe depending on the location of the tissue to be treated or critical structures to be avoided. In another embodiment, the probe includes at least one activatable end region carried on the distal region of the probe. It will be appreciated that the electrodes or activatable end regions may be activated by application of electrical, RF, or microwave current applied to a conductive material such as an electrode or an antenna, current may be applied to a resistive heating element (tip or electrode). In other embodiments, ultrasound-generating current may be applied to an ultrasound generator or sonicator tip, a cryogenic fluid may circulated through a circulation pathway through a lumen of the probe or electrode, described herein, or in a tip, or an ablative fluid, e.g., ethanol or high salt, may be ejected from the end of a needle tip. In one embodiment the fluid can be injected into or near a target tissue to modify the conductivity of the target tissue near the electrode. In one aspect, the agent can be an iso-tonic or hyper-tonic solution. Such solutions can increase the local conductivity of the target tissue. Alternatively, a hypo-tonic solution or D5W (5% dextrose in water) solution can be infused into the target tissue to reduce the local conductivity of the target tissue.

In one exemplary embodiment, the activatable end regions are RF or microwave or electroporation electrodes or antennas. It will be appreciated that the elongate probe may further utilize a combination of activating methods, such as, but not limited to RF, microwave, IRE, cryoablation, supraporation, or other types of ablation methods. “Supraporation” uses much higher voltages and currents, in comparison to electroporation, but with shorter pulse widths.

The at least one electrode may be two or more electrodes for bipolar electrode configurations and/or an array of electrodes (either bipolar or monopolar). The electrodes can be operated in monopolar or bipolar modes, and may be capable of switching between the two modes. The electrodes can be coupled to a power supply and/or a ground pad electrode, in monopolar mode, via an insulated wire which can be a guidewire. The coupling can also be made via a coaxial cable, thereby allowing for coupling of one or both electrodes to the power supply as a ground pad electrode. In some exemplary embodiments, the electrodes are coupled to the power supply such that power may be independently applied to each electrode. The electrodes may be independently coupled to the power supply where the power supply has independent channels, or the electrodes may be coupled to a multiplexer that controls power to each of the electrodes separately.

The electrodes can be made of a variety of conductive materials, both metallic and non-metallic. Suitable materials for the electrode include, in non-limiting embodiments, steel such as 304 stainless steel of hypodermic quality, platinum, gold, silver and alloys and combinations thereof. Also, the electrodes can be made of conductive solid or hollow straight wires of various shapes such as round, flat, triangular, rectangular, hexagonal, elliptical, and the like. In a specific embodiment all or portions of electrodes can be made of a shaped memory metal, such as NiTi, commercially available from Raychem Corporation, Menlo Park, Calif. A radiopaque marker can be coated on the electrodes for visualization purposes.

The electrodes can be coupled to the probe using soldering, brazing, welding, crimping, adhesive bonding and other joining methods known in the medical device arts.

In one embodiment, the apparatus further comprises an elongate sleeve 112 integral with or carried on the distal end region of the probe. The sleeve preferably comprises an elongate tubular barrel having a proximal region 118 and a distal region 116. The sleeve is preferably open at the proximal end for receiving and engaging at least part of the distal end region of the probe. The sleeve includes at least one opening 120 in the distal end region. In one exemplary embodiment, the sleeve includes a plurality of openings in the distal end region. Where the apparatus includes deployable electrodes or antennas, the electrodes are aligned with the openings such that the electrodes are deployable through the openings. In one embodiment, the sleeve partially houses the probe, forming therewith, a chamber that communicates with the openings and the proximal region of the sleeve. The sleeve and/or the probe may include a marker system for connecting the sleeve and the probe such that the electrodes are aligned with the openings and deploy through the openings. As seen in FIG. 1A, the probe may include a knob or slide 23 for deployment of the electrodes 22. In the embodiment shown, the electrodes are deployed when the knob is moved towards the distal end of the probe. As the knob is moved toward the proximal end of the probe, the electrodes are retracted within the device.

At least a portion of the sleeve and/or probe can be made from a variety of resilient polymers including elastomers, polyesters, polyimides, fluoropolymers and the like. The sleeve can be configured to be both electrically and/or thermally insulative or can be electrically and/or thermally conductive using conductive polymers known in the art. An example of a conductive polymer includes Durethane™ C manufactured by the Mearthane Products Corporation (Cranston, R.I.). The sleeve can further be formed of a conductive material such as stainless steel, nickel, platinum, and/or aluminum. It will be appreciated that different portions of the sleeve and/or probe may be made of different materials.

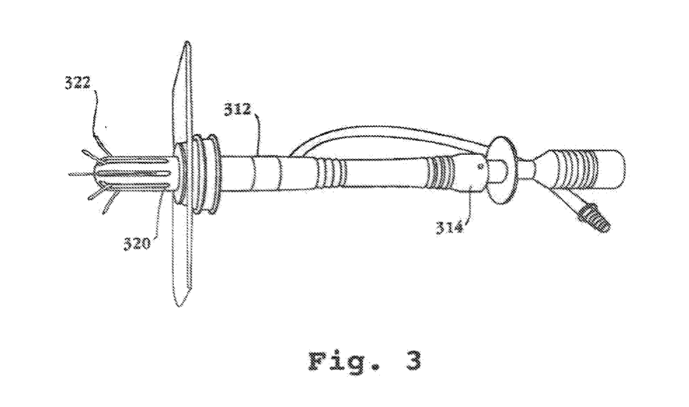

The sleeve can be made to any suitable shape and size depending on the length of the ablation device and/or the depth/width of the cavity. Suitable shapes include, but are not limited to, cylindrical, ellipsoid, football shaped, etc. In one preferred embodiment, the sleeve is an elongate, tubular barrel. The sleeve preferably includes a cylindrical distal portion adapted to at least partially house and engage the distal region of the ablation device. As seen in FIG. 3, the sleeve 312 preferably includes a cylindrical distal portion including at least one opening 320 configured to receive at least one electrode 322 of the ablation device 314. In one exemplary embodiment, the sleeve has a non-tissue piercing distal end.

The opening(s) may be microporous or include a covering to prevent tissue from clogging the opening(s). In another embodiment, the openings may include a plurality of openings sized to prevent clogging from the tissue. In yet another embodiment, the openings include a tissue filter 13 positioned on the outer or inner surface of the openings or covering to prevent clogging of the openings with tissue. In one embodiment, the filter is a perforated, meshed, or weaved membrane sized to allow the electrodes 22 to deploy therethrough. In another embodiment, the opening may be covered by a porous material such as a plastic or gel that the electrodes pierce when deployed through the openings. In yet another embodiment, the openings may be covered by a mesh or screen where the electrodes deploy through the mesh screen. The mesh may be a spiral mesh. The mesh screen may be formed of any suitable material including, as non-limiting examples, metals such as stainless steel or brass, polyester, nylon, and fiberglass. In one exemplary embodiment, the mesh is formed from nylon monofilament fiber. The mesh may be any suitable mesh including, but not limited to, a welded, monofilament, or perforated mesh.

In another embodiment, the apparatus further includes a tubular sheath or covering 123 surrounding at least a portion of the distal region of the sleeve and at least partially positioned over the at least one opening. The sheath may further be affixed to the sleeve. The sheath is preferably a semi-porous or porous membrane or mesh. The sheath is preferably low profile and sized to prevent interference with the movement of the apparatus or deployment of the electrodes. The sheath may be formed of any suitable material that allows penetration of the electrodes. Preferably, the material is semi-porous or porous. In another embodiment, the material is made porous by mechanical means such as stamping or piercing. Exemplary materials include plastics and polymers such as silicon, Dacron™, and ethylene vinyl acetate (EVA).

As shown in FIG. 1A, the apparatus 10 may further include a thermally insulative barrier, shield, or heat skirt 11 positioned around the distal end portion 17 of the probe. The barrier is preferably positioned distal to the plate 24. This barrier serves to limit the thermal effect of the activatable region. In this manner, the ablation area may be contained and/or skin burns minimized. The barrier may be formed of any suitable low thermal conductivity material. Non-limiting examples include ceramic, foam, and plastics such as polyetherimide (Ultem). The barrier may be any suitable shape as needed for the cavity such as elliptical, oval, circular, cone-shaped, frustoconical, etc. In another embodiment, the barrier includes an internal air or liquid chamber to allow air flow through the barrier to provide cooling to protect critical structures such as the skin. In yet another embodiment, the barrier is configured to allow for circulation of air and/or a liquid for cooling. In a non-limiting example, the barrier includes an internal spiral chamber with at least one intake opening whereby air may be pumped into or drawn into the chamber to circulate therethrough. The barrier may also include an opening for the air and/or liquid to exit. In the embodiment where the probe includes a vent, the air or liquid may be drawn out of the cavity through the vent. In another embodiment, the air or liquid is recirculated through the barrier and/or a cooling system. Also shown are the proximal portion of the probe 21 and the tubing connector 26 for connection to the suction source.

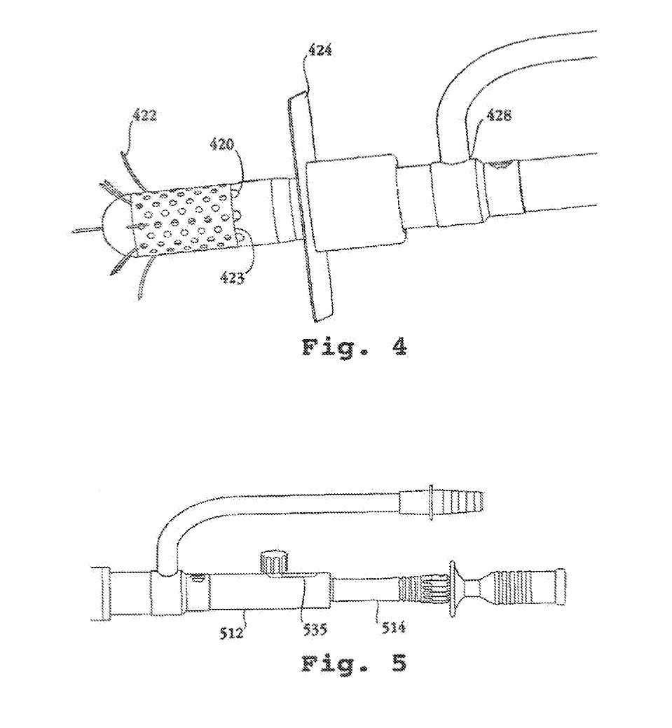

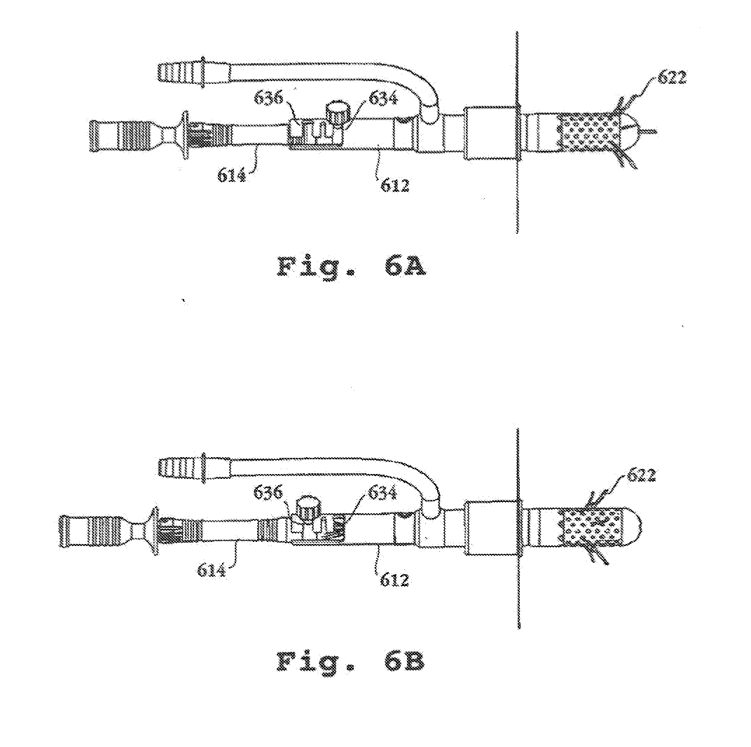

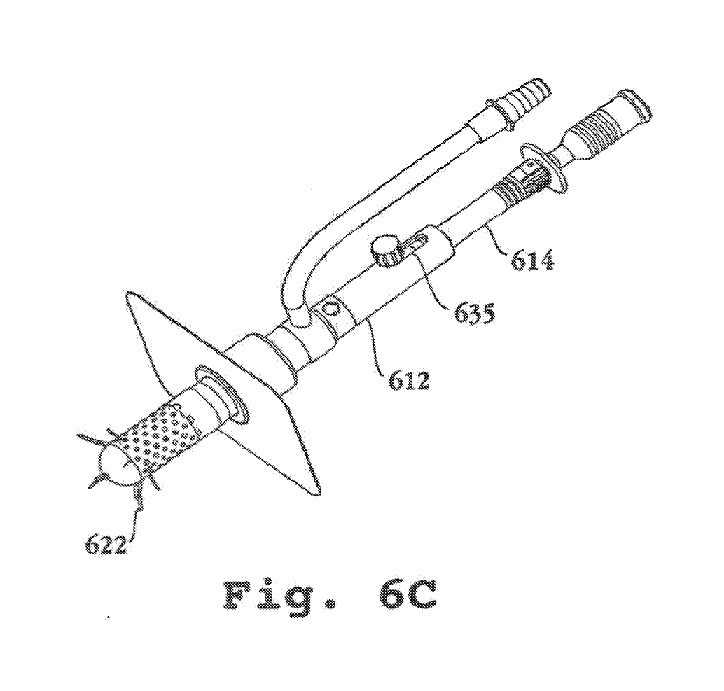

In one embodiment, the sleeve is affixed to the probe through any suitable means such as a clip, lock, or other fastener. In the embodiment seen in FIG. 5, the fixture is a linear slot 527 in the proximal portion of the sleeve 512, whereby at least a portion of the probe 514 is positioned in the slot to provide alignment of the electrodes with the openings when the electrodes (622 in FIGS. 6A-6B) are deployed. At least a portion of the probe is slidably positioned in the slot, where the maximum proximal and distal axial movement of the probe within the sleeve is determined by the length of the slot. In another embodiment, as seen in FIGS. 6A-6B, the fixture is a lock positioned in the sleeve 612 including multiple slot positions 634, 636 for receiving at least a portion of the probe 614. In yet another embodiment, not shown, the sleeve is not affixed to the probe and is, instead, axially slidable along the distal region of the probe. In a further embodiment, the sleeve is integral with or fixed to the probe.

In one exemplary embodiment, suction can be applied at the opening(s) by applying a vacuum to the proximal end region of the sleeve to draw tissue adjacent or against the sleeve or collapse the cavity against the sleeve allowing ablation of the tissue drawn against or adjacent the surface of the sleeve when a vacuum is applied to the opening(s). In one embodiment, the sleeve includes at least one port 128 for connection to a source of suction. Port 128 may be, but is not limited to, a luer fitting, a valve (one-way, two-way), a toughy-bourst connector, a swage fitting, and other adaptors and medical fittings known in the art. The connection is also referred to herein as connecting structure, and may include tubing, fittings, couplings, or any fastening suitable for providing suction therethrough from a suction source to the apparatus. The suction source may be connected to the sleeve through any suitable connector as exemplified by standard ¼ inch medical suction tubing and fittings 126. The suction source may be the standard suction available in the operating room and may be coupled to the device using a buffer. In other embodiments, suction can be applied from a conventional vacuum generator such as a vacuum pump, a venture vacuum generator powered by pressurized air or water supply, or an external vacuum unit. It will further be appreciated that any suitable suction source may be used with the device including, but not limited to, a vacuum pump or the standard surgery vacuum. The amount of suction applied to the apparatus is non-limiting as long as the suction is sufficient to draw the walls of the cavity adjacent the sleeve. Typically, the suction is provided at a negative pressure of about 0 to about 736 mm Hg. It will be appreciated that the settings for vacuum pressure may vary depending on the tissue type, size of the cavity, and the age, health, and body type of the patient. In one embodiment, the suction is suitable to retain the apparatus substantially vertical to the surface of the treatment tissue.

In another embodiment, the distal-most end of the sleeve or probe is at least partially open forming a nozzle at the distal end of the probe. When suction is applied to the apparatus, the tissue is drawn into the nozzle. In this embodiment, electrodes may be positioned at the interior of the nozzle or the distal end of the probe may be conductive for ablation of the tissue drawn into the nozzle. It will be appreciated that the distal end of the probe may be concave or tubular shaped to allow for drawing the tissue therein. It will be appreciated that this embodiment is particularly useful for cysts, polyps, as well as any other tissue that may be isolated in this manner for ablation.

The apparatus may include a seal disposed between the sleeve and the probe to prevent flow of air between the sleeve and the probe. Any suitable sealing member may be used including, but not limited to, an o-ring, gasket, or flange.

The apparatus may further include a vacuum control valve or port 15, 115 for regulation of the amount of vacuum obtained at the opening(s). In one embodiment, the valve is an on/off valve such that when the valve is in the open position, air is drawn from the valve opening on the sleeve whereby little or negligible vacuum is achieved at the opening(s) in the sleeve distal region. When the valve is in the closed position, the vacuum is achieved at the opening(s) in the sleeve distal region. In another embodiment, the apparatus may include a vacuum control, not shown, as known in the art to regulate the amount of vacuum achieved. In another embodiment, the apparatus includes an overflow relief valve whereby air is allowed to enter the cavity when an excess of suction is applied to the cavity. The vacuum control may be manually or automatically operated.

The apparatus may further include a sealing member or plate 124 disposed at the distal region 116 of the sleeve. The plate is adapted to be pressed against a patient's surgical site or target tissue when the probe is inserted into the surgical cavity formed in the patent, to cover and seal the opening of said cavity or the surface of the target tissue. The covering allows for a more efficient vacuum to be applied to the surgical cavity or the target tissue to draw the tissue to the sleeve. It will be appreciated that the size of the plate is non-limiting as long as the plate is sized to at least cover the opening of the surgical cavity or the surface of the target tissue. It will be appreciated that the plate may be adjusted or cut down in accord with the size of the surgical opening. The plate can be constructed from rigid polymers such as polycarbonate or ABS or resilient polymers including Pebax polyurethane, silicones HDPE, LDPE, polyesters and combinations thereof. In one exemplary embodiment, the plate is formed of a pliable or compliant material. It will be appreciated that the sealing plate may be formed of a transparent, semi-transparent, or opaque material. Where the plate is formed of a transparent material, the cavity may be monitored for wrinkles, dimples, pockets, etc., which can indicate an air pocket, and/or incorrect or incomplete suction of the tissue to the probe. The sealing plate may be made in any suitable shape for covering the cavity opening and contacting the tissue surface, including, but not limited to circular, oval, elliptical, rectangular, and square. Where the plate is conformable, the plate may be any suitable thickness that allows the plate to conform to the tissue surface, yet is resilient enough to resist being drawn into the cavity. In an exemplary embodiment, the plate is formed of silicone having a thickness of about 0.076 inches. In one embodiment, at least one face of the sealing plate includes a conformable surface that conforms or bends to the shape of the tissue surface. This can be accomplished by constructing all or a portion of the plate from resilient polymers including, but not limited to, elastomers such as silicone and polyurethane and polymers thereof as well as foam rubber. The plate can be fabricated from such materials using injection molding or mandrel dip coating methods known in the art. One or both surfaces of the plate may further be coated with an agent that improves contact with the skin and/or assists in the formation of the seal. In another embodiment, the plate may be treated to impart desired properties to the plate. An exemplary coating is a slippery or lubricous agent coated on the tissue contact surface of the plate to prevent the skin adhering to the plate. A non-limiting example is a plate that is plasma treated on the tissue contact surface to provide a lubricous surface. A preferred example is a silicone plate that is plasma treated on the tissue contact surface. The sealing plate may be a solid plate, include one or more plate sections, or include baffles or passages to allow air flow between two or more plates. In this manner, the tissue surface may be cooled to prevent or reduce the occurrence of burns. The plate may further include a marker 252 or markings to externally indicate the extent of the ablation margin. For example, the plate may have an indicator to show the extent of ablation based on the deployment of the electrodes.

In one embodiment, the sealing plate is axially slidable along the proximal region of the sleeve. It will be appreciated that shallow ablations (generally less than 1 cm) may cause burning or blistering of the skin. Accordingly, in one embodiment, the apparatus includes means on the sleeve for limiting the axial movement of the sealing plate toward the sleeve's distal end to prevent skin burns. Exemplary means for limiting axial movement include a stop or resistive gradient on the sleeve or the plate. In another embodiment, markers 250 can be disposed along the sleeve to facilitate identification of the location of the probe distal end within the sleeve. In this manner, the surgeon can position the sealing plate to allow at least about 1 cm between the sealing plate and the distal end of the probe within the sleeve.

In another embodiment, the sealing plate comprises at least one port for connection to the suction source, not shown. In this embodiment, the vacuum in the cavity is created by applying suction to the sealing plate port. It will be appreciated the suction may be applied at the sealing plate port alone or in conjunction with applying suction at the at least one opening in the sleeve.

The sealing plate may further be formed of a conductive material, where the plate acts as a ground pad electrode. In this embodiment, the plate may be directly connected to the power source, or may be connected through the apparatus. In this embodiment, the sealing plate should be of a sufficient size to provide adequate dissipation of current to prevent burns. In another embodiment, the sealing plate may be hollow or comprise an area for conductive air flow to dissipate heat. The hollow plate may also be connected to the suction source to facilitate and enhance heat dissipation by conductive air flow.

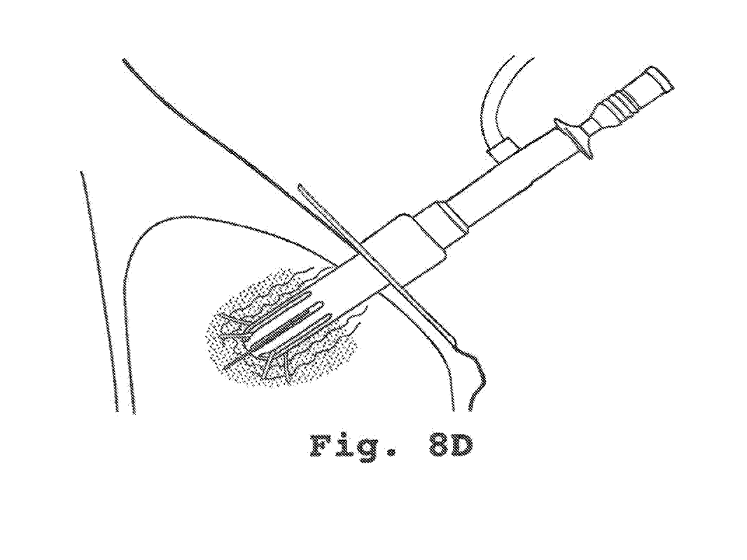

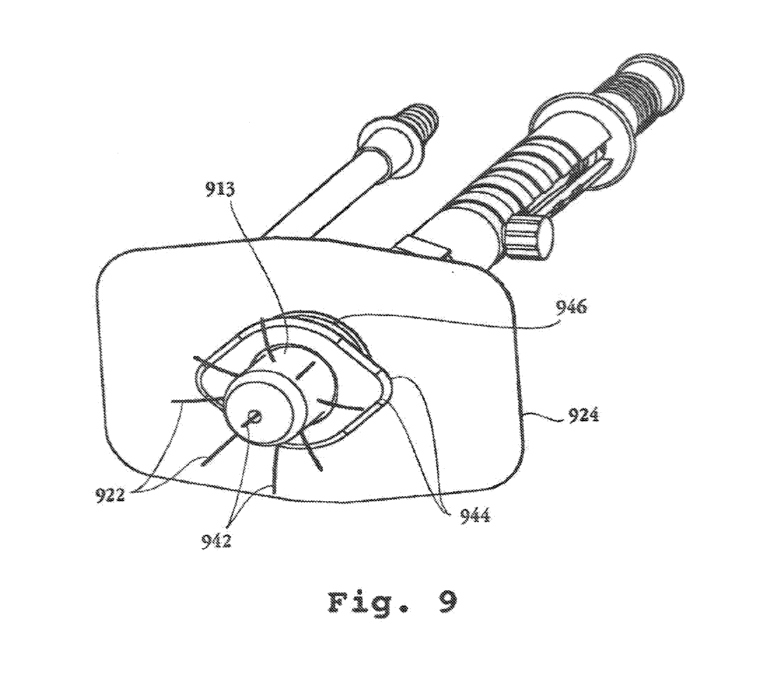

As shown in FIG. 9, the apparatus may further comprise at least one temperature sensor positioned at least one of (i) on the sealing plate 924 for measuring the temperature at the surface of the surgical cavity, (ii) on the sleeve 913 for sensing temperature within the surgical cavity, and/or (iii) on one or more surfaces of the thermal barrier 944. Where the sensor(s) is positioned on the thermal barrier, it will be appreciated that sensor(s) positioned on the distal side of the barrier or the area of the probe or sleeve distal to the barrier (see 19 in FIG. 1A), where used, will approximately measure the temperature of the tissue being ablated. Sensor(s) positioned on the proximal side of the barrier or the proximal portion of the probe or sleeve will approximately measure the temperature of the tissue cavity that is not ablated. In this manner, skin burns can be minimized and/or prevented. The sensor may be any suitable thermal sensor. The apparatus may further include a temperature indicator positioned on the sealing plate. This indicator may include thermotropic liquid crystals that change position according to changes in temperature. The liquid crystals can be calibrated as a visual indication of a desired temperature or end point for the ablation. In another embodiment, at least one sensor is positioned on the sealing plate operatively connected to the indicator for sensing and indicating temperature at the surface of the surgical cavity. In another embodiment, the apparatus includes a temperature sensor positioned on the sleeve and operatively connected to the indicator for sensing temperature within the surgical cavity. At least one of the electrodes 922 may also include a thermal sensor 942. It will be appreciated that all or some of the electrodes may include a thermal sensor. In a particular embodiment, alternating electrodes include a thermal sensor. Thermal sensors can include thermistors, thermocouples, resistive wires, optical sensors and the like. A suitable thermal sensor includes a T type thermocouple with copper constantene, J type, E type, K type, fiber optics, resistive wires, thermocouple IR detectors, and the like. It will be appreciated that the control of power to the electrodes may be adjusted or controlled based on feedback from the at least one thermal sensor. The feedback may be a closed-loop whereby a feedback signal is received at a control or the energy source, which then regulates the amount of energy or current delivered to electrodes. In another embodiment, the power may be manually regulated based on feedback from the at least one thermal sensor.

As seen in FIGS. 2A-2B, the sleeve may be an adapter 212 for use with an ablation device 214 of the type having (i) an elongate probe having a distal region 230 and a proximal end region and (ii) one or more electrodes disposed at the probe's distal end region, for ablating tissue when electrical, radiofrequency or microwave power is applied to the electrode(s). Exemplary ablation devices include the Starburst XL™ and NanoKnife® generator and probes (AngioDynamics, Inc., Latham, N.Y.).

A variety of activation devices, including energy-delivery devices such as power sources, can be utilized by embodiments of the invention. Specific energy delivery devices and power sources that can be employed in one or more embodiments include, but are not limited to, the following: (i) a microwave power source adapted to be coupled to a microwave antenna distal end tip, providing microwave energy in the frequency range from about 915 MHz to about 2.45 GHz (ii) a radio-frequency (RF) power source adapted to be coupled to a distal end electrode, (iii) a reservoir containing heated fluid adapted to be coupled to a catheter with a closed or at least partially open lumen configured to receive the heated fluid, (iv) a reservoir of a cooled fluid adapted to be coupled to a catheter with a closed or at least partially open lumen configured to receive the cooled fluid, e.g., a cryogenic fluid, (v) a resistive heating source adapted to be coupled to a conductive wire distal-end structure, (vi) an ultrasound power source adapted to be coupled to an ultrasound emitter tip, wherein the ultrasound power source produces ultrasound energy in the range of about 300 kHz to about 3 GHz, and (vii) combinations thereof. In one embodiment, the power source can be a RF energy source such as the 1500X RF generator (AngioDynamics, Inc., Latham, N.Y.), which delivers 1-250 W at 460 kHz. The 1500X RF generator provides temperature control of 15° C. to 125° C.±3° C. In yet another embodiment, the power source can be a NanoKnife® generator (AngioDynamics, Inc., Latham, N.Y.).

In one exemplary embodiment, the energy delivery device can be an RF power supply that provides RF current to one or more RF electrodes. In several exemplary embodiments, the RF power supply delivers electromagnetic energy in the range from 5 to 200 watts to the electrodes at about 450 V although it will be appreciated that wider ranges of energy delivery levels may be possible with different power supplies as well as with different configurations. The electrodes are coupled to the energy source either directly to each electrode, or indirectly using a collet, sleeve, connector, cable and the like which couples one or more electrodes to the energy source. Delivered energies can be in the range of 1 to 100,000 joules, with embodiments having ranges of approximately 100 to 50,000 joules, 100 to 5000 joules, and 100 to 1000 joules. Lower amounts of energy can be delivered for the ablation of smaller structures such as nerves and small tumors as well as higher amounts of energy for larger tumors. Also delivered energies can be modified (by virtue of the signal modulation and frequency) to ablate or coagulate blood vessels vascularizing the tumor. This provides for a higher degree of assurance of ablation of the blood supply of the tumor.

The adapter includes at least one, or a plurality of, opening(s) 220 in the distal end region of the adapter. In operation, the electrodes of the ablation device are aligned with the openings such that the electrodes deploy through the openings into the target tissue. The adapter may comprise a tubular sheath 223 surrounding the distal region of the sleeve and at least partially positioned over the at least one opening. The sleeve may include a port or connector 228 for connection to a source of suction. The suction source may be connected to the port through any suitable connection such as tubing 226 and fittings 232. The port may further be connected to the distal region of the sleeve through an internal passageway. The sleeve may further include a vacuum control valve or port 215 as further described above.

In one embodiment, the adapter includes a sealing member or plate 224 disposed on the distal region of the sleeve. The sealing plate may comprise a planar cover and a ring slidable around the tubular distal region of the sleeve. In one exemplary embodiment, the sealing member is positioned distal of port 228. The planar cover may be pliable, rigid or semi-rigid. The planar cover may further include at least one face with a convex, concave, flat, or substantially flat surface. As noted above, the cover and/or the ring of the sealing plate are constructed from rigid polymers such as polycarbonate or ABS or resilient or flexible polymers including Pebax®, polyurethane, silicones HDPE, LDPE, polyesters and combinations thereof. The sealing member may be integral with or connected to the sleeve. It will be appreciated the sealing member may further include a port, not shown, for connection to a suction source.

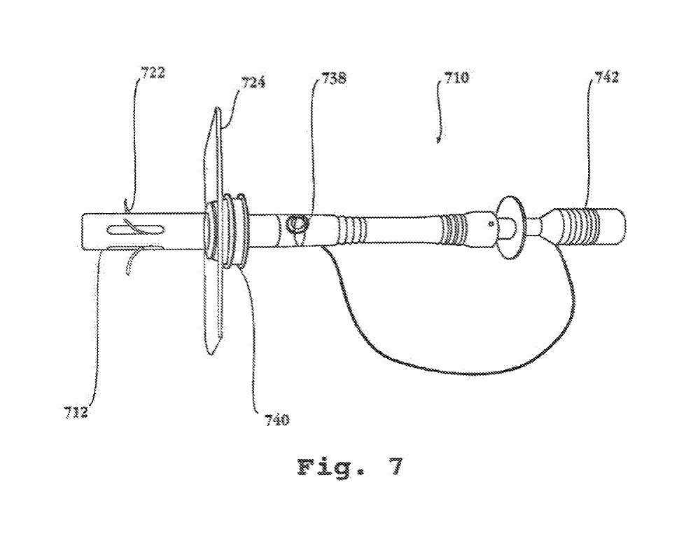

As seen in FIG. 7, in another embodiment, the apparatus 710 includes an activatable distal end 712 formed of a conductive material. The apparatus may include one or more deployable electrodes or non-conductive probes for thermal sensing 722. The distal end includes one or more openings operatively connected to a suction port 738. When suction is applied, the cavity walls are drawn to the distal end of the apparatus. The distal end is activated to ablate a margin of tissue surrounding the distal end forming an ablated margin. The apparatus may further include a sealing plate 724 to cover and/or seal the opening of the surgical cavity. The sealing plate may be connected to the apparatus by a flexible baffle 740 such that the plate is pressed securely against the skin surface. In one embodiment, an actuator 742 is used to retract and deploy the electrodes or non-conductive probes.

### III. Method of Using Cavity Ablation Device

The following discussion pertains particularly to the use of an RF energy source and treatment/ablation apparatus. For purposes of this discussion, the activatable distal ends are referred to as RF electrodes/antennas and the energy source is an RF energy source. However it will be appreciated that all other energy delivery devices and sources discussed herein are equally applicable, such as, but not limited to, the NanoKnife® generator and probes (AngioDynamics, Inc.). It will be appreciated that any RF generator capable of delivering power in the required range is suitable for use in the present method including, but not limited to, the EPT-1000 TCT™ RF generator (Boston Scientific, Natick, Mass.), the S-270RF generator (Electropulse, Russia), and the Cool-Tip™ Generator (Valley Lab, Boulder, Colo.).

In another aspect, the invention includes a method of ablating margins of a surgical cavity formed in a tissue. The surgical cavity is generally a tumor bed where a tumor and margin of healthy tissue have been excised by the treating worker, e.g., physician. The surgical cavity is generally a tubular, cylindrical, or “football” shaped hole with at least one opening at the skin. The method includes ablating the vertical walls and/or the bottom of the cavity.

Once a tumor lesion is removed, the physician inserts the apparatus at least partially into the surgical cavity. The apparatus is preferably manipulated to place the tip of the instrument at or near the bottom of the cavity. If the apparatus has one or more deployable electrodes, the apparatus is usually inserted into the cavity with the electrodes in a retracted state. The position of the apparatus with respect to the target area can be confirmed by conventional imaging techniques, as further described below. As seen in FIG. 4, in one embodiment, a sealing plate 424 or adjustable cover may be axially adjusted along the sleeve to position the plate against the tissue surface thereby to seal the surgical cavity and assist creating the vacuum in the cavity. When suction is applied, the tumor bed collapses against the surface of the apparatus.

A suction source connected to the apparatus at port 428 is used to apply suction at distal surface regions of the apparatus and create a vacuum in the cavity, thereby to draw at least a portion of the cavity wall into contact with the apparatus. Once the apparatus is so positioned, the electrode(s) 422 are deployed through the openings 420 at the sleeve distal region and piercing the sheath 423. As indicated above, the electrodes, and particularly deployable electrodes, can be shaped such that in the deployed state they form a desired geometric configuration. In one embodiment, the electrodes are independently deployed from the probe distal end. It will be appreciated that all or a portion of the electrodes may be deployed with different shapes or to different lengths. It will further be appreciated that not all of the electrodes may be deployed, especially where an asymmetric ablation is desired. The electrodes may further be deployed a variable distance from the sleeve to create the appropriate margin. The electrodes may be deployed to a desired depth in the tissue, or may be deployed step-wise to a maximum depth while delivering power. Specific margins to be ablated include 0.5 cm, 1 cm, 1.5 cm, or more. In one embodiment, a plurality of electrodes are deployed into the cavity walls at radially spaced intervals that, with activation of the electrodes define an ablation volume surrounding the apparatus and form the ablated margin.

Preferably, while suction is maintained the electrodes are activated to ablate surrounding tissue and create an ablated margin. In one embodiment, this step involves applying an RF current to one or more electrode structures carried on or deployed from the probe distal region. Power and duration levels for application of RF current are detailed above. Typically, ablation is carried out up to a target temperature and held at the target temperature to allow heat dissipation through to the tissue surrounding the electrode surface. The target may be a selected temperature, e.g., 100° C. or greater, a selected temperature over a given time period, e.g., 45° C. to 100° C. for a period or 5-20 minutes, or a rapid increase in impedance. It will be appreciated that the ablation endpoint may be adjusted based the tissue ablated, the size of the cavity, etc. A typical ablation in breast tissue is ablating the tissue at 100° C. for 15 minutes. A typical endpoint is a thermal dose or time at a specified temperature. Both the temperature and the time may be dependent on the tissue being ablated.

As seen in FIG. 5, the sleeve 512 may include a linear slide 527 for receiving at least a portion of the ablation device 514. In this manner the sleeve and device are engaged such that the electrodes, when deployed, are aligned with the openings in the sleeve. As seen in FIG. 6C, the linear slide 627 is positioned at the proximal portion of the sleeve 612 and preferably includes a section for entry of at least a portion of the probe 614 into the slide area. In this manner, the probe may be axially adjusted within the sleeve by movement of the probe within the slide. Deployment of the electrodes 622 from the openings in the sleeve may be reciprocally adjusted. It will be appreciated that the length of the slide may be varied based on several factors such as the length of the openings as well as the depth of the cavity.

As seen in FIGS. 6A-6B, the sleeve 612 may further include a locking mechanism with two or more slots 634, 636 for receiving a portion of the device 614. The mechanism is configured to allow the physician to selectively control the amount of the probe housed in the sleeve, and thus the position of the deployment of the electrodes within the openings. In use, the physician first locks the probe in the distal-most slot 634. As seen in FIG. 6A, in this configuration, the electrodes are deployed from the distal region of the openings. This position can be used to ablate a margin of tissue near the bottom of the cavity. For longer cavities, the surgeon can then retract the electrodes and reposition the probe 614 to the proximal locking slot 636. As seen in FIG. 6B, the electrodes 622 are then deployed from a proximal region of the openings in the sleeve. The apparatus may include a plurality of slots 634, 636 to provide a range of deployment of the electrodes through the openings. It will be appreciated that the physician may position the sleeve using the slots in any sequence.

It will be appreciated that the electrodes may be deployed radially or asymmetrically depending on the position of the cavity and surrounding structures. In this manner, a variety of different geometries, not always symmetrical, can be ablated. For example, for cavities near the chest wall or other critical structures, the electrodes may be deployed to ablate only the vertical walls, or a portion thereof.

The method can further utilize, before and/or after the tumor is excised, known imaging systems such as X-ray graphs, computerized tomography, MRI, scintigraphy, or ultrasound imaging to locate one or more specific tumor areas of interest and, optionally, to map the extent of the tumor lesion.

The temperature of the tissue, the device, or of the electrodes may be monitored, and the output power of the energy source adjusted accordingly. Temperature can be maintained to a certain level by having a feedback control system adjust the power output automatically to maintain that level. The physician can, if desired, override the closed or open loop system.

The closed loop system can also utilize a controller to monitor the temperature, adjust the RF power, analyze the result, refeed the result, and then modulate the power. More specifically, the controller governs the power levels, cycles, and duration that the RF energy is distributed to the electrodes to achieve and maintain power levels appropriate to achieve the desired treatment objectives and clinical endpoints. The controller can be integral to or otherwise coupled to the power source. The controller can be also be coupled to an input/output (I/O) device such as a keyboard, touchpad, PDA, microphone (coupled to speech recognition software resident in the controller or other computer) and the like. After a cool-down cycle of about 30 seconds, the sensors positioned on the electrode or the sleeve may be used as an indicator of the temperature of the tissue in the feedback process. In another embodiment, a feedback control system can be operatively connected to the energy source, the at least one sensor, and the electrodes. The feedback control system receives temperature data from the sensor(s) and the amount of electromagnetic energy received by the electrodes is modified from an initial setting of ablation energy output, ablation time, temperature, and current density (the “Four Parameters”) based on the data received from the sensor(s). In one embodiment, the feedback control system can automatically change any of the Four Parameters. The feedback control system may include a multiplexer (digital or analog) to multiplex different electrodes, sensors, sensor arrays, and/or a temperature detection circuit that provides a control signal representative of temperature or impedance detected at one or more sensors. A microprocessor can be connected to the temperature control circuit.

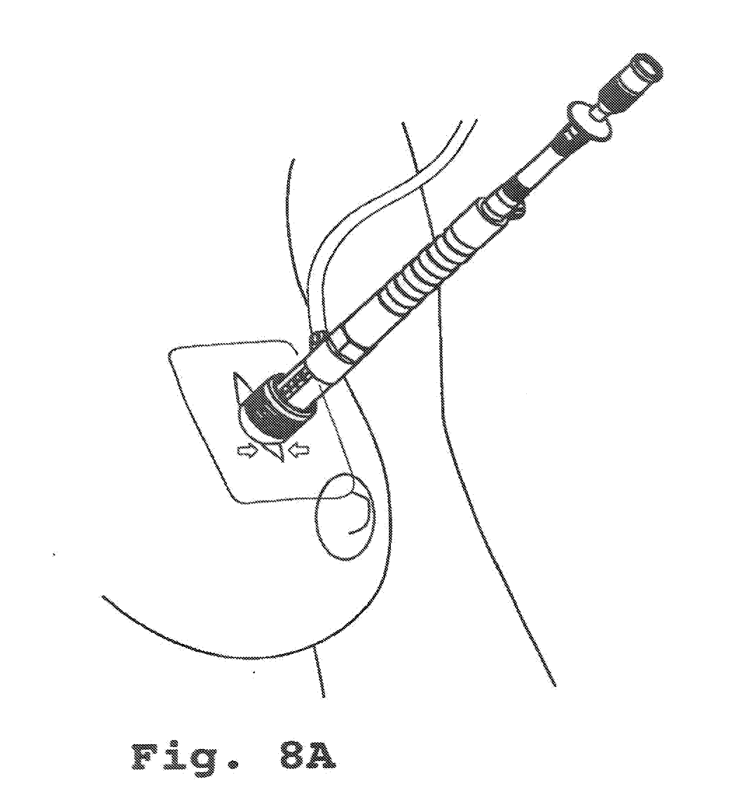

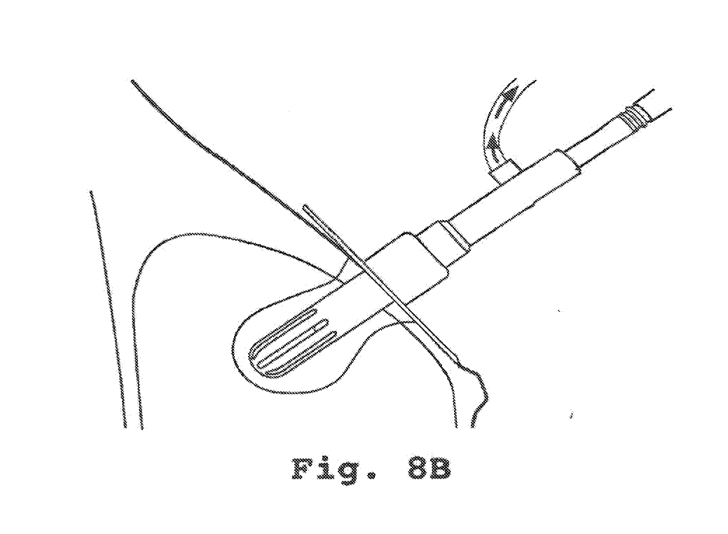

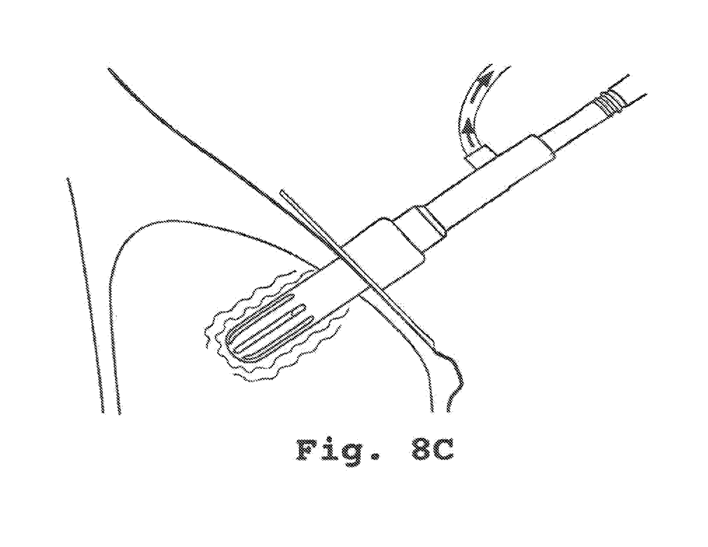

As seen in FIGS. 8A-8D, the method of the invention is well suited for ablation of surgical cavities in the breast, especially lumpectomy cavities. According to the ACS, the use of breast-conserving surgeries now accounts for nearly ½ of all the breast cancer surgeries performed in the U.S. each year. There are approximately 150,000-170,000 lumpectomies performed per year in the US. In this method, the apparatus is positioned at least partially in the lumpectomy cavity (FIG. 8A). In one exemplary embodiment, the distal end of the apparatus is placed at or near the bottom of the surgical cavity. The sealing plate may be axially adjusted on the sleeve or probe such that the sealing plate is adjacent the surface of the breast. Suction is then applied to the suction port at the sleeve and/or plate, which creates a vacuum in the cavity (FIG. 8B). Application of suction is effective to draw the tissue surrounding the cavity into contact with the surface of the sleeve (FIG. 8C). At least one electrode is deployed from the apparatus through at least one opening in the sleeve distal region and activated to ablate a margin of tissue surrounding the surgical cavity (FIG. 8D). The electrode(s) are retracted and the device is withdrawn from the cavity. It will be appreciated that before withdrawing the device from the cavity, the electrodes may be deployed at different axial positions within the cavity to achieve a margin of ablated tissue along a desired length of the cavity. This is especially useful for longer cavities.

With Breast Conserving Surgery (BCS) the tumor is removed along with a variable margin of tissue, usually about 1 cm, surrounding the tumor. The margin is then assessed for malignant cells. If there are no malignant cells in the margin (negative margin), the surgery is considered to have removed all cancerous tissue. The majority of the breast is left intact, and depending on the amount of tissue spared, cosmetic results are usually satisfactory.

However, if the margin includes malignant cells near the tissue edge (close margin) or even at the tissue edge (negative margin), the patient must endure a second surgery to remove more tissue.

Using the present method, the need for additional surgery is reduced or eliminated. Another benefit of the present method is a known margin of ablated tissue at least partially surrounding the cavity. This ablated margin is usually in addition to the margin of tissue resected around the tumor. As detailed in Example 1, the apparatus was used to ablate a cavity formed in breast tissue. Briefly, the apparatus was placed in a cavity formed in breast tissue obtained from a mastectomy. The sealing member was adjusted to a position adjacent the tissue surface and suction was applied to the sleeve at the port. The tissue was drawn against the sleeve with no voids visible upon inspection through the sealing plate. The electrodes were deployed into the tissue surrounding the cavity and activated to form a margin of ablated tissue surrounding the cavity. After the procedure, the tissue appeared to be necrosed and coagulated, indicating the tissue was successfully ablated.

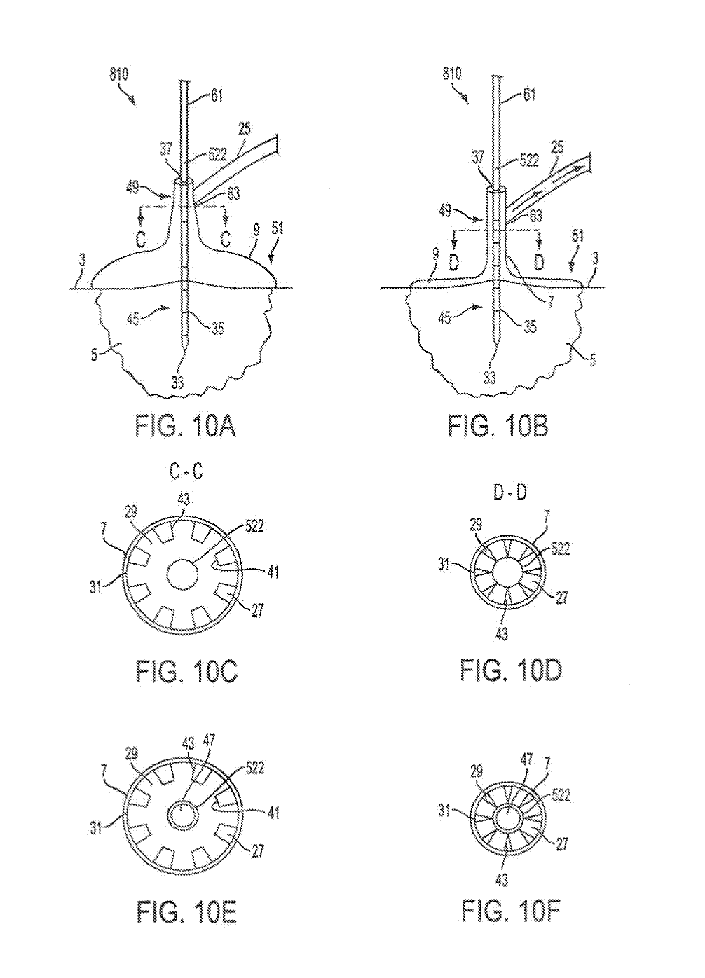

In another aspect, the method may be used for asymmetric ablation of a cavity wall, or a particular area of a cavity wall. In this embodiment, the cavity may be a surgical cavity or a body cavity. In this embodiment, the apparatus includes a tissue contacting surface adapted to be placed adjacent or against the treatment site. In one embodiment, the apparatus may be thermally insulated to reduce or prevent ablation of undesired tissue in the cavity. The tissue contacting surface may further include a deployable structure such as a suction port or suction cup 9 (FIGS. 10A and 10B) for affixing the tissue contacting surface to the treatment site or holding the treatment site to the tissue contacting surface of the apparatus. In this manner the tissue may be fixed to the apparatus and held stable for insertion of the electrodes to the target tissue. At least one electrode is deployed from the tissue contacting surface into the tissue to be treated and activated to produce an ablated tissue margin surrounding the at least one electrode. In this manner, the cavity wall can be selectively ablated. It will be appreciated this embodiment may be useful for treatment of esophageal cancer, uterine fibroids, cysts, a tumor with a necrotic core, and colon polyps, among others. It will be appreciated that the device may be used in any body, target tissue, or surgical cavity where ablation of the cavity margins is desired. In another embodiment, the device may be used for ablation of a tubular cavity such as a vessel or duct. For this embodiment, the device may further include a deployable or inflatable section or structure distal to the activating structure to seal the cavity. An exemplary structure is an expandable balloon that can be deployed distal to the probe to seal the tubular cavity such that suction can be used to draw the tissue of the tubular cavity to the probe. The device may also include a barrier at the proximal end of the probe to seal the tubular cavity proximal to the probe. In an exemplary embodiment, the apparatus may include a deployable stent at the distal end for use in treating an aneurysm. In this embodiment, the probe is deployed into the vessel and at least one barrier is deployed distal to the probe distal end to seal the vessel. Suction is used to draw the tissue of the tubular cavity to the probe and the electrode(s) are deployed and activated to collapse the aneurysm. The probe is removed leaving the stent in situ. It will be appreciated that the stent may become affixed to the cavity tissue during ablation and/or a suitable adhesive may be used to affix the stent to the tissue site.

### EXAMPLES

The following example illustrates but is in no way intended to limit the invention.

### Example 1

### Ablation of Margins in Breast Tissue

A section of tissue was excised from breast tissue obtained from a mastectomy. The apparatus was positioned in the cavity such that the distal end of the sleeve contacted the bottom of the cavity. The sealing plate was adjusted adjacent the tissue surface and suction was applied using a surgical suite available vacuum supply. The walls of the cavity were drawn against the distal end of the sleeve and no voids were visible through the sealing plate. Electrodes were deployed into the tissue and activated to a target temperature of 100° C. After 15 minutes at the target temperature, the tissue was visually inspected and determined to be coagulated and necrosed. Indicators of tissue necrosis included a visual change in the coloration and texture of the tissue and/or a temperature above 70° C. 30 seconds after cease of electrode activation. It will be appreciated that other methods of visualizing cell death are suitable including the use of dyes and stains that have phallic properties to dead cells.

Another embodiment of an ablation device is described herein with reference to FIGS. 10A and 10B. FIG. 10A illustrates a probe assembly 810 which comprises an elongate probe 522 having a distal region 45 and a proximal region 61. In one embodiment, the probe can comprise one electrode 522, as illustrated. The probe 522 can include at least one activatable end region carried on the distal region 45 of the probe. In one aspect, the electrode 522 can be comprised of a conductive material made of metal, such as, but not limited to stainless steel or other conductive materials. The electrode 522 can have a sharp needle tip 33 capable of piercing tissue. At least one radiopaque marker 35 can be coated on the probe 522 for visualization purposes.

As illustrated in FIGS. 10A and 10B, at least a portion of the outer surface of the electrode or probe 522 can be substantially and completely surrounded by a conformable deployable structure 9. The structure 9 can be adapted to conform to a surface of the skin 3 or the target tissue 5. The structure 9 can be, but is not limited to, a skirt, cone, barrier, a suction cup, or other deployable structure, at least a portion of which is capable of being attached and fixed to a target tissue surface 3. In one aspect, the deployable structure 9 can comprise at least one opening 37 at the proximal end 49 of the deployable structure 9. The at least one opening 37 can be positioned around the distal end portion 45 of the probe 810 and can be configured for selective receipt of the probe 522. The deployable structure 9 can be attached to and axially slidable along at least a portion of the outer surface of electrode 522. The deployable structure 9 has an outer surface 7 and an inner surface 31, a proximal portion 49, and a distal portion 51. At least a portion of the distal portion 51 of the deployable structure 9 is capable of forming a seal between the deployable structure 9 and the outer surface of the skin 3 when vacuum suction is applied to the surface 3 of the skin 3 through vacuum hose 25.

Deployable structure 9 can be symmetrical, asymmetrical, elliptical, oval, circular, conical, or at least partially frustoconical in shape. Alternatively, the deployable structure 9 may be made of any suitable shape for contacting a target tissue surface, including, but not limited to circular, oval, elliptical, rectangular, and square. In one embodiment, at least a portion of the deployable structure 9 includes a conformable surface that conforms or bends to the shape of the tissue surface 3. The conformable deployable structure may be any suitable thickness that allows the deployable structure to conform to the target tissue surface 3, yet is resilient enough to resist being drawn into target tissue. In one embodiment, as illustrated, the proximal portion 49 of the deployable structure 9 can be smaller in diameter and size compared to the distal portion 51 of the deployable structure 9 when the structure 9 is in an unbiased position, such as illustrated in FIG. 10A. If RF energy is used to treat the target tissue 5, deployable structure 9 can serve to limit the thermal effect of the activatable region. In this manner, the ablation zone may be contained.

A hollow space or chamber 29 inside of the deployable structure 9 can be defined between the skin surface 3 and the inner surface of the shield 31. As illustrated, the probe 522 is capable of being deployed into target tissue 5 through chamber 29. In one exemplary embodiment, although not illustrated, the probe assembly 810 can further comprise a means that is attached to and axially slidable along the outer surface of the probe 522 for limiting the axial movement of the deployable structure 9 toward the probe's distal end. Exemplary means for limiting axial movement can include a stop or resistive gradient on the surface of the probe 522. In another embodiment, markers 35 can be disposed along the probe 522 to facilitate identification of the location of the probe distal end.

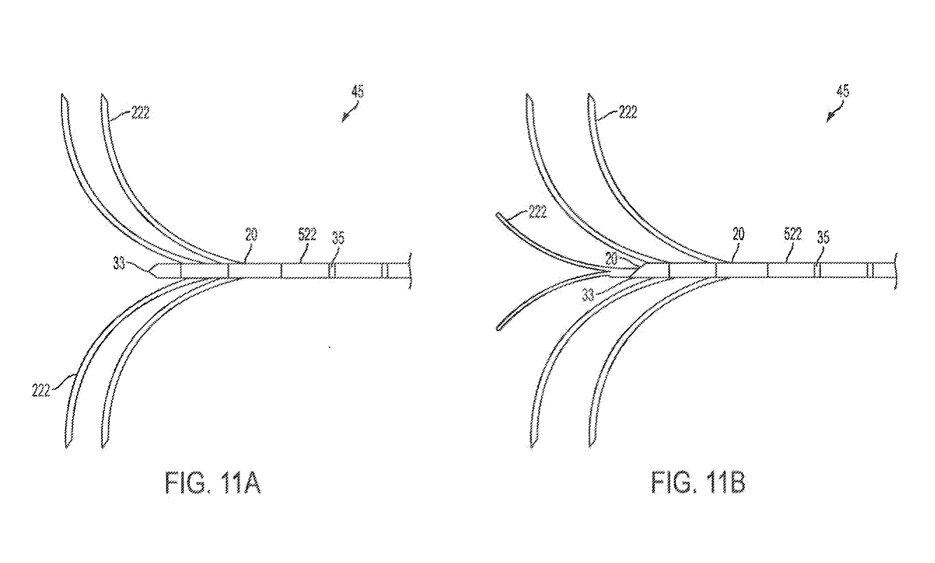

In an alternative embodiment, electrode 522 can comprise a lumen 47, illustrated in FIGS. 10E and 10F. In one aspect, lumen 47 can extend substantially the entire length of the electrode probe 522 to a solid tip 33, as illustrated in FIGS. 10A and 11A. Alternatively, lumen 47 can extend substantially all the way through the probe 522 to a distal opening 20, as illustrated in FIG. 11B. The lumen 47 can be capable of receiving suction air from vacuum suction hose 25 (FIGS. 10A and 10B). Additionally, the lumen 47 is capable of receiving a plurality of additional electrodes 222. In yet another embodiment, lumen 47 is capable of receiving at least one agent such as a fluid or liquid for infusion into a target tissue 5 or cystic lesion either before, during, or after delivery of RF energy, microwave energy, or the delivery of electrical pulses sufficient to irreversibly electroporate at least a portion of target tissue 5. Vacuum suction can be applied from the vacuum suction hose 25 through the inner lumen 47 of the electrode and into the at least one opening 20 positioned at the distal end of the electrode 522, as described below. The addition of a lumen 47 extending along the longitudinal axis of the probe 522 may require that the probe 522 be larger in size compared to a probe without a lumen. The larger probe 522 size can provide an advantage in that it can allow for a larger RF or IRE ablation area and a greater resistance to impeding out when performing an RF ablation.

As illustrated in FIG. 10B, in a non-limiting example, the probe device 522 can comprise at least one port 63 for connection to a source of suction. Port 63 may be, but is not limited to, a luer fitting, a valve (one-way, two-way), a toughy-bourst connector, a swage fitting, and other adaptors and medical fittings known in the art. The connection can also referred to herein as connecting structure, and may include tubing, fittings, couplings, or any fastening suitable for providing suction therethrough from a suction source to the apparatus. The suction source may be connected to the deployable structure 9 through any suitable connector as exemplified by standard ¼ inch medical suction tubing and fittings. The suction source may be the standard suction available in the operating room and may be coupled to the device using a buffer. In other embodiments, suction can be applied from a conventional vacuum generator (not shown) such as a vacuum pump, a venture vacuum generator powered by pressurized air or water supply, or an external vacuum unit. It will further be appreciated that any suitable suction source may be used with the device including, but not limited to, a vacuum pump or the standard surgery vacuum. The amount of suction applied to the apparatus is non-limiting as long as the suction is sufficient to draw the walls of the target tissue 5 adjacent the probe 522. Typically, the suction is provided at a negative pressure of about 0 to about 736 mm Hg. It will be appreciated that the settings for vacuum pressure may vary depending on the tissue type, size of the cavity, and the age, health, and body type of the patient. In one embodiment, the suction is suitable to retain the probe assembly 810 substantially vertical to the surface of the target tissue 5, as illustrated in FIGS. 10A and 10B.

The suction source connected to the apparatus 810 at port 63 can be used to apply suction at distal surface regions of the probe assembly 810 to create a vacuum in the target tissue, thereby to draw at least a portion of the target tissue 5 wall into contact with the probe assembly 810. Preferably, while the electrodes are activated to deliver electrical energy to the target tissue 5 to ablate surrounding tissue and create an ablated margin, suction is maintained. Alternatively, suction can be applied before or after electrical energy is delivered to the target tissue.

In one embodiment, the applied electrical energy can be an RF current, microwave energy, or electrical pulses sufficient to product reversible or irreversible electroporation of the target tissue 5, to one or more electrode structures carried on or deployed from the probe distal region 45. Parameters for application of the RF current and IRE electrical pulses are described herein. It will be appreciated that the RF or IRE ablation endpoint may be adjusted based the tissue ablated, the size of the ablated tissue, or the size of the body cavity or target tissue 5. Both the temperature and the time may be dependent on the tissue being ablated. When IRE pulses are delivered to the target tissue 5, any combination of the number of pulses, the amplitude, and the duration of the pulses may be adjusted to achieve the desired ablation margins.

Vacuum air may be pumped into or drawn into the lumen or chamber 29 to circulate therethrough. The air can be suctioned out through the vacuum hose 25, as illustrated by the arrows in FIG. 10B. Although not shown, in other embodiments, the deployable structure 9 may also include an additional opening for the air and/or liquid to exit. The probe 522 can also include a vent, and the air or liquid may be drawn out of the target tissue through the vent. In another embodiment, the air or liquid can be recirculated through a barrier and/or a cooling system. When suction is applied through hose 25 into the lumen 29 of the deployable structure 9, as is described below, the shape of the deployable structure 9 can change such that the deployable structure 9 can go from an unbiased state to a biased state. In one aspect, the structure 9 can be smaller in size in the biased position compared to the un-biased position, as illustrated in FIGS. 10A and 10B. In one aspect, at least a portion of the deployable structure 9 can be capable of conforming to the surface 3 of the skin, as described above. In the biased state the deployable structure 9 can closely adhere to the surface of the skin 3. Similar to other embodiments described herein, in one exemplary embodiment, suction can be applied at the opening(s) 20 of the probe 522 by applying a vacuum to the proximal end region of the probe 522.

The deployable structure 9 can be comprised of a biocompatible, conformable plastic material. All or a portion of the deployable structure 9 can be manufactured from resilient polymers including, but not limited to, elastomers such as silicone, polyurethane, and polymers thereof as well as foam rubber or polyetherimide (Ultem). In an exemplary embodiment, the deployable structure can be formed of silicone having a thickness of about 0.076 inches. The deployable structure 9 can be fabricated from such materials using injection molding or mandrel dip coating methods known in the art. Any of the inner or outer surfaces of the deployable structure 9 may further be coated with an agent that improves contact with the skin and/or assists in the formation of the seal between the deployable structure 9 and the skin surface 3. In another embodiment, the deployable structure 9 may be treated to impart desired properties to the deployable structure. An exemplary coating is a slippery or lubricous agent coated on the tissue contact surface of the deployable structure 9 to prevent the skin adhering to the structure 9. A non-limiting example is a deployable structure 9 that is plasma treated on the tissue contact surface to provide a lubricous surface. One exemplary embodiment is a silicone deployable structure that is plasma treated on the tissue contact surface.

In one embodiment, illustrated in FIGS. 10C through 10F, one or more ribs 27 can be attached to or integral with at least a portion of the inner surface 31 of the deployable structure 9. Each rib 27 can have at least three outwardly-facing surfaces and one surface that can be attached to the inner surface 31 of the deployable structure 9. One of the three outwardly facing surfaces 41 faces the outer wall of the electrode 522, while two of the surfaces 43 are side-facing surfaces that face adjacent ribs 27. Each rib 27 can be separated or spaced from an adjacent rib 27 by a gap. When suction is applied through suction hose 25 into the lumen 29 of the deployable structure 9, the curved or slightly arcuate shapes of the outer face 41 of each of the ribs 27 can circumferentially surround the outer surface of the electrode 522, illustrated in FIGS. 10D and 10F. The internal ribs 27 serve to preserve a vacuum passage through the lumen 29 when the inner faces 41 of the ribs 27 abut against the outer surface of the probe 522, as illustrated in FIGS. 10D and 10F. In one aspect, gaps between each rib 27 can allow room for each of the ribs 27 to fully surround the outer surface of the electrodes 522 without crowding when suction is applied through the vacuum hose 25. In one embodiment, the plurality of internal ribs 27 can be attached to or integral with the inner surface 31 of the deployable structure 9 from below the opening 63 where the suction tube 25 is connected to the deployable structure 9 at the proximal end 49 of the deployable structure 9 to the distal end 51 of the deployable structure 9. In one embodiment, although not illustrated in FIGS. 10A and 10B, at least a portion of the inner surface 31 of the deployable structure 9 can be substantially covered with the plurality of ribs 27. The ribs 27 can be made of the same material as the deployable structure 9. The ribs 27 can be any suitable size or shape for adhering to the tissue surface 3.

Referring to FIGS. 11A and 11B, in one exemplary embodiment, the probe 522 can have at least one opening 20 positioned at the distal end 45 of the probe 522 along the outer surface of the probe. Deployable electrodes 222 can extend within a portion of the probe 522 and can be aligned with the openings 20 of the probe 522 such that the electrodes are deployable through the openings 20. The electrodes 222 can be made of a variety of conductive materials, both metallic and non-metallic. Suitable materials for the electrode include, in non-limiting embodiments, steel such as 304 stainless steel of hypodermic quality, platinum, gold, silver and alloys and combinations thereof. Also, the electrodes can be made of conductive solid or hollow straight wires of various shapes such as round, flat, triangular, rectangular, hexagonal, elliptical, and the like. In a specific embodiment all or portions of electrodes 222 can be made of a shaped memory metal, such as NiTi, commercially available from Raychem Corporation, Menlo Park, Calif. At least one radiopaque marker 35 can be coated on the electrodes for visualization purposes. The electrodes can be coupled to the probe using soldering, brazing, welding, crimping, adhesive bonding and other joining methods known in the medical device arts.

If the apparatus 810 has one or more deployable electrodes, the probe assembly is usually inserted into the target tissue with the electrodes 222 in a retracted state. The position of the probe assembly 810 with respect to the target tissue 5 can be confirmed by conventional imaging techniques. The additional electrodes 222 are deployable from the distal end of the probe 522 for ablating a target tissue 5 when activated. It will be appreciated that the electrodes 222 may be deployed radially or asymmetrically from the probe 522 depending on the location of the target tissue 5 to be treated or critical structures to be avoided. In one aspect, the electrodes 222 or activatable end regions may be activated by application of electrical energy for reversible or irreversible electroporation of the target tissue, RF, or microwave current to the target tissue 5.

Once the probe assembly 810 is so positioned, the electrode(s) 222 can be deployed through the openings 20 at the probe distal region 45. As indicated above, the electrodes, and particularly deployable electrodes, can be shaped such that in the deployed state they form a desired geometric configuration. In one embodiment, the electrodes are independently deployed from the probe distal end. It will be appreciated that all or a portion of the electrodes may be deployed with different shapes or to different lengths. It will further be appreciated that not all of the electrodes may be deployed, especially where an asymmetric ablation is desired. The electrodes may further be deployed a variable distance from the probe 522 to create the appropriate margin. The electrodes 222 may be deployed to a desired depth in the target tissue 5, or may be deployed step-wise to a maximum depth while delivering power. Specific margins to be ablated include 0.5 cm, 1 cm, 1.5 cm, or more. In one embodiment, a plurality of electrodes 222 can be deployed into the target tissue 5 at radially spaced intervals that, with activation of the electrodes 222 define an ablation volume surrounding the apparatus and form the ablated margin.

A method of using the devices, particularly those described in FIGS. 10A through 11B, will now be described. After a lesion or target tissue 5 has been detected and the location determined using ultrasound or fluoroscopic imaging, at least one probe 522, as illustrated in FIGS. 10A and 10B, can be inserted into the patient's skin 3 such that the probes 522 can be near to or in contact with the target tissue 5. Electrical connectors (not shown) from each probe 522 can be connected to a power source, such as a generator, using an extension cable. This completes an electrical circuit between the electrodes 522 and the generator. Although a single probe 522 is illustrated, more than one probe 522 can be inserted into a patient's skin 3 during the procedure.

When the probe 522 is inserted through the skin 3 into the target tissue 5, at least a portion of electrode 522 can be surrounded by deployable structure 9. The deployable structure 9 can be axially adjusted along the outer surface of the electrode 522, as necessary, before or during the treatment procedure. This design allows for minimal friction between the outer surface of the electrode 522 and the inner surface of the deployable structure 9 and ease of movement of the electrode 522 into the target tissue 5 in relation to the deployable structure 9 during insertion and use of the electrode 522 in the target tissue 5. As illustrated in FIGS. 10A and 10C, after the probes 522 have been inserted into the target tissue 5, no vacuum suction is applied through the vacuum hose 25 that is connected to deployable structure 9, and the deployable structure 9 remains in a relaxed or non-biased position.

As illustrated in FIGS. 10B and 10D, when suction is applied through the suction hose 25 from a vacuum source, as described above, at least a portion of the deployable structure 9 can substantially completely surround the electrode 522 such that the inner face 41 of the ribs 27 surrounds at least a portion of the outer surface of the electrode 522, and the chamber 29 between the outer surface of the skin 3 and the inner surface 31 of the deployable structure 9 is decreased compared to the unbiased position of the deployable structure 9 when no suction is applied to the lumen 29 of the device.

In another embodiment, as illustrated in FIGS. 10E and 10F, suction can be applied from a vacuum source through vacuum hose 25 to an interior portion of the probe 522, for example, through lumen 47 of the electrode 522, into at least one opening 20 of the probe 522 at the distal end 45 of the probe after the distal end 45 of the probe is placed within a target tissue 5. Suction is then applied to the suction port 63, which creates a vacuum in the target tissue 5 (FIG. 10B). Application of suction is effective to draw at least a portion of the target tissue 5 into contact with the probe 522. Before or during the application of electrical energy and/or suction, at least one electrode 222 can be deployed from the probe assembly 810 through at least one opening 20 in the probe distal region and activated to ablate a margin of tissue surrounding the target tissue, as illustrated in FIGS. 11A and 11B. The electrode(s) can then be retracted and the probe assembly withdrawn from the target tissue.

In yet another embodiment, after the probe 522 is inserted into the target tissue 5, suction can be applied through the opening 20 of the distal end 45 of the probe 522 such that the suction air delivered from a suction source contacts the target tissue 5. The force from the suction air against the target tissue 5 is capable of drawing the target tissue 5 onto at least a portion of the outer surface of the electrode 522 and/or onto at least a portion of the openings 20 to hold a portion of the target tissue 5 against the opening 20. Once a portion of the target tissue 5 is stabilized in place against the openings 20, electrical energy such as RF or electrical pulses such as IRE pulses can be delivered to the target tissue 5 sufficient to irreversibly electroporate the target tissue 5 or to deliver radiofrequency energy to the target tissue 5. This method provides an advantage for treating tissues for which it would normally be difficult to obtain good contact between the target tissue 5 and the outer surface of the electrode 522.

In one aspect, a probe 522, such as that illustrated in FIGS. 10A and 10B can be inserted into a tubular bodily structure, such as a bowel, intestine, esophagus, vessel, or other tubular structure, or a cystic lesion in which it would normally be difficult to get good or sufficient contact between the target tissue 5 and the electrodes 522, 222, and suction can be applied, as described above, to draw a portion of target tissue 5 into contact with internal electrodes 222 that are positioned or aligned within the openings 20. In yet another aspect, the method of applying suction between a target tissue 5 and/or cystic lesion against an opening 20, as described herein, can be used to stabilize the outer surface of the target tissue 5 or cystic lesion against the outer surface of the ablation device 522.

Electrical pulses can be applied simultaneously while suction is being applied across the electrodes 522 in the desired pattern to electroporate the cells of the target tissue, creating field gradient lines (not shown) sufficient to non-thermally electroporate the target tissue. Alternatively, electrical pulses can be applied before or after suction through the chamber 29 of the deployable structure 9. After treatment, extension cables can be disconnected from the electrical connector and the probes 522 removed from the target tissue.

In one embodiment, the method of ablating a cystic lesion is described herein. Before electrical pulses are delivered to the cystic lesion, the target tissue 5 in which a cystic lesion is located can be pre-treated using RF ablation or IRE ablation using any of the parameters described herein. If RF ablation is used, however, the thermal heating caused by the RF ablation may not produce an even ablation result, and the target tissue 5 or the cystic lesion may be unevenly heated or ablated. A cystic lesion could be present anywhere in a patient's body. The cystic lesion can comprise cancerous cells. It can also contain a mixture of live tissue and dead tissue. If any portion of the target tissue or cystic lesion is unevenly heated as a result of RF ablation, at least a portion of the target tissue 5 or cystic lesion may be incised, and undesirable material inside or near the cystic lesion can be withdrawn through an interior portion of the probe 522, such as lumen 47, using the suction methods described herein. Other undesirable material within or near the cystic lesion can contain a cancerous mesh of fluid, diseased tissue, and malignant tissue. All undesirable tissue, such as cancerous, liquid, liquid-like, or fluid tissue, or other diseased or malignant tissue can be suctioned or withdrawn from the cystic lesion using vacuum air from the vacuum source. Any remaining target tissue 5 or cystic lesion may or may not comprise all or a portion of the intended cystic lesion for ablation. The remaining target tissue 5 can be put in contact with the probe 522 to be incised, and any desired part of the remaining target tissue 5 can be incised. Following this step, the remaining portions of the target tissue 5 or cystic lesion to be ablated can be put in contact with the one or more electrodes 522, 222, power can be turned on, and electrical energy, such as RF energy or electrical pulses for electroporation, can be delivered to ablate the cystic lesion or target tissue 5.

In other embodiments, a cryogenic fluid may circulated through the lumen 47, a circulation pathway in the probe 522, the tip 33, or an ablative fluid, e.g., ethanol or high salt, may be ejected from the end of a needle tip. The suction can be applied to a cystic lesion or a lesion with a necrotic core, and the suction can be used to draw out liquid from the necrotic core or cystic lesion. The cystic lesion can become a natural cavity, i.e., a space that could be evacuated of liquid. Fluid or undesirable material from the cystic lesion can be withdrawn through an interior portion of the probe, such as the lumen 47, using the suction means disclosed herein. In yet another embodiment, any other desired fluid, such as, but not limited to saline or D5W can be ejected from the electrode, such as at the needle tip.

In yet another method, any of the devices and methods described herein can be used to collapse a vascular aneurism. This can be done by applying low energy RF energy to shrink collagen surrounding and aneurysm, thereby reducing the aneurysm. In some instances, this can be followed by application of suction, as described herein, to remove any remaining cellular debris.

The devices and methods described herein can be applied using reversible or irreversible electroporation. Example embodiments for reversible electroporation can involve using any of the devices described herein to deliver to a target tissue 1-8 electrical pulses with a field strength of 1-100 V/cm. Other embodiments altering cellular structures adversely involve generators having a voltage range of 100 kV-300 kV operating with nano-second pulses with a maximum field strength of 2,000 V/cm to and in excess of 20,000 V/cm between electrodes. Certain embodiments can involve between 1-15 pulses between 5 microseconds and 62,000 milliseconds, while others can involve pulses of 75 microseconds to 20,000 milliseconds. In certain embodiments the electric field density for the treatment is from 100 Volts per centimeter (V/cm) to 7,000 V/cm, while in other embodiments the density is 200 to 2000 V/cm as well as from 300 V/cm to 1000 V/cm. Yet additional embodiments have a maximum field strength density between electrodes of 250V/cm to 500V/cm. The number of pulses can vary. In certain embodiments the number of pulses can be from 1 to 100 pulses. In one embodiment, as described herein, between about 10 pulses and about 100 pulses can be applied at about 2,000 V/cm to about 3,000 V/cm with a pulse width of about 10 μsec to about 50 μsec. After applying these pulses, a predetermined time delay of from about 1 second to about 10 minutes can optionally be commenced in order that intra-cellular contents and extra-cellular contents of the target tissue cells can mix. This procedure can be repeated, as necessary, until a conductivity change is measured in the tissue. Following this step, about 1 pulse to about 300 pulses of about 2,000 V/cm to about 3,000 V/cm can be applied with a pulse width of about 70 μsec to about 100 μsec to widely ablate the tissue. This last step can be repeated until a desired number of ablation pulses is delivered to the tissue, for example, in the range of about 10 pulses to about 300 pulses, more particularly, about 100 pulses. In other embodiments, groups of 1 to 100 pulses (here groups of pulses are also called pulse-trains) are applied in succession following a gap of time. In certain embodiments the gap of time between groups of pulses can be from about 0.5 second to about 10 seconds.

Therapeutic energy delivery devices disclosed herein are designed for tissue destruction in general, such as resection, excision, coagulation, disruption, denaturation, and ablation, and are applicable in a variety of surgical procedures, including but not limited to open surgeries, minimally invasive surgeries (e.g., laparoscopic surgeries, endoscopic surgeries, surgeries through natural body orifices), thermal ablation surgeries, non-thermal surgeries, as well as other procedures known to one of ordinary skill in the art. The devices may be designed as disposables or for repeated uses.

In yet another embodiment, the electrodes can be adapted to administer electrical pulses as necessary in order to reversibly or irreversibly electroporate cells of a target tissue. By varying parameters of voltage, the number of electrical pulses, and pulse duration, the electrical field will either produce irreversible or reversible electroporation of the target tissue 5. The pulse generator of the present invention can be designed to deliver a range of different voltages, currents and duration of pulses as well as number of pulses. Typical ranges include but are not limited to a voltage level of between 100 and 3000 volts, a pulse duration of between 20 and 200 microseconds (more preferably 50-100 microseconds), and multiple sets of pulses (e.g. 2-5 sets) of about 2-25 pulses per set and between 10 and 500 total pulses. The pulse generator can administer a current in a range of from about 2,000 V/cm to about 6,000 V/cm. The pulse generator can provide pulses which are at a specific known duration and with a specific amount of current. For example, the pulse generator can be designed upon activation to provide 10 pulses for 100 microseconds each providing a current of 3,800 V/cm+/−50%+/−25%, +/−10%, +/−5%. The electroporation treatment zone is defined by mapping the electrical field that is created by the electrical pulses between two electrodes.

When electrical pulses are administered within the irreversible parameter ranges, as described above, permanent pore formation occurs in the cellular membrane, resulting in cell death of the target tissue. Alternatively, electrical pulses may be administered within a reversible electroporation range. Temporary pores will form in the cellular membranes of target tissue cells.

The voltage pulse generator can be configured to generate electrical pulses between the electrodes in an amount which is sufficient to induce irreversible electroporation of cells of the target tissue. Specifically, the electrical pulses can create permanent openings in cells of the target tissue 5, for example, thereby invoking cell death without creating a clinically significant thermal effect. The target tissue cells will remain in situ and can be subsequently removed by natural body processes.

In one embodiment, the electroporation pulses can be synchronously matched to specifically repeatable phases of the cardiac cycle to protect cardiac cellular functioning. See, for example, U.S. Patent Application No. 61/181,727, filed May 28, 2009, entitled “Algorithm For Synchronizing Energy Delivery To The Cardiac Rhythm”, which is fully incorporated by reference herein. This feature is especially useful when the electroporation pulses are delivered in a location that is near the heart. In one aspect, electroporation pulses can be synchronized with a specific portion of the cardiac rhythm. Electrocardiogram (ECG) leads (not shown) can be adapted to be attached to the patient for receiving electrical signals which are generated by the patient's cardiac cycle. The ECG leads transmit the ECG electrical signals to an electrocardiogram unit. The electrocardiogram unit can transmit this information to a synchronization device which can include hardware or software to interpret ECG data. If the synchronization device determines that it is safe to deliver electroporation pulses, it sends a control signal to a pulse generator. The pulse generator can be adapted to connect to the electrical connector for delivering electroporation pulses. Each of the synchronization device and pulse generator can be implemented in a computer so that they can be programmed.

It will be appreciated that embodiments described with respect to one aspect may be applicable to each aspect of the device and method described. As a non-limiting example, the thermal barrier may be used with the elongate sleeve as well as with an integral probe. It will further be appreciated that embodiments may be used in combination or separately. It will also be realized that sub-combinations of the embodiments may be used with the different aspects. Thus, although embodiments have been described with many optional features, these features are not required unless specifically stated.

It will also be realized that the apparatus may be used in combination with other procedures or methods as appropriate. For example, the apparatus may be used in conjunction with chemotherapy, surgery, and/or a thermally activated therapeutic agent.

The foregoing description provides specific details for an understanding of, and enabling description for, embodiments of the apparatus. However, one skilled in the art will understand that the invention may be practiced without these details. In other instances, well-known structures and functions have not been shown or described in detail to avoid unnecessarily obscuring the description of the embodiments of the invention.

1. A method for electrically ablating a target tissue, comprising:
positioning a probe assembly in or near the target tissue to be ablated, wherein the probe assembly comprises a probe having a proximal end, a distal end, and an outer surface; and a deployable structure having an inner surface, wherein at least a portion of the deployable structure surrounds at least a portion of the outer surface of the probe;
positioning the deployable structure near the target tissue;
applying suction to at least a portion of the probe assembly; and
applying electrical energy to the probe to electrically ablate the tissue.

2. The method of claim 1, wherein the method further comprises axially adjusting the deployable structure along the outer surface of the probe before positioning the deployable structure, wherein the deployable structure is one or more of a shield, a barrier, a skirt, a suction cup, and a cone.

3. The method of claim 1, wherein during the step of positioning the deployable structure, the method further comprises positioning at least a portion of the deployable structure against a surface of a patient's skin.

4. The method of claim 1, wherein the deployable structure further comprises a plurality of ribs positioned on the inner surface, and a chamber, wherein during the step of applying suction, the suction is applied to the chamber.

5. The method of claim 4, wherein during the step of applying suction to the chamber, the deployable structure transitions from an unbiased state to a biased state, wherein the plurality of ribs surrounds the outer surface of the probe in a contacting relationship.

6. The method of claim 4, further comprising forming an air-tight seal between at least a portion of the deployable structure and a surface of a patient's skin during the step of applying suction.

7. The method of claim 1, wherein at least one opening is positioned at the distal end of the probe, and wherein the method further comprises applying suction at the at least one opening of the positioned probe to draw at least a portion of target tissue into contact with the at least one opening of the probe.

8. The method of claim 7, wherein at least one deployable electrode is aligned with the at least one opening, and wherein during the step of applying suction, at least a portion of target tissue is drawn into contact with the at least one electrode.

9. The method of claim 1, wherein the method further comprises stabilizing the deployable structure and the probe relative to the target tissue during the step of applying suction.

10. The method of claim 1, wherein the target tissue comprises one or more of a cyst, cancerous tissue, uterine fibroids, a tumor with a necrotic core, a polyp, a lesion, a vessel, a duct, an aneurysm, and a body cavity.

11. The method of claim 10, wherein the method further comprises removing at least a portion of the target tissue through suction, wherein the target tissue comprises a liquid.

12. The method of claim 1, further comprising applying electrical pulses to the probe in an amount which is sufficient to induce irreversible electroporation of cells of the target tissue tissue, but which is insufficient to induce thermal damage to substantially all of the target tissue such that the identified tissue cells are killed by irreversible electroporation.

13. The method of claim 1, further comprising applying electrical energy as one or more of radiofrequency energy and microwave energy.

14. The method of claim 1, further comprising infusing at least one agent into the target tissue before, during, or after the step of applying suction, or any combination thereof, wherein the agent is selected from the group comprising: saline, D5W, and a hypo-tonic solution.

15. The method of claim 1, wherein the method further comprises before applying suction, applying electrical energy to ablate at least a portion of the target tissue.

16. The method of claim 14, wherein the method further comprises applying electrical energy in the form of a sufficient number of electrical pulses to irreversibly electroporate the target tissue.

17. The method of claim 14, wherein the method further comprises applying electrical energy as one or more of radiofrequency energy and microwave energy.

18. The method of claim 1, wherein the method further comprises during the step of applying electrical energy to the target tissue, maintaining the suction.

19. The method of claim 1, wherein the probe comprises a lumen extending along a longitudinal axis of the probe, and wherein suction is applied through the lumen of the probe.

20. The method of claim 1, wherein the probe comprises a lumen extending along a longitudinal axis of the probe, and wherein before, during, or after the step of applying electrical energy, or any combination thereof, at least one agent is infused into the lumen of the probe, wherein the agent is selected from the group comprising: saline, D5W, and a hypo-tonic solution.

21. A method for electrically ablating a target tissue, comprising:
positioning a probe in or near a tissue to be ablated, wherein the probe comprises a proximal end and a distal end, wherein at least one opening is positioned at the distal end of the probe, and wherein at least one electrode is aligned with the at least one opening;
applying suction at the at least one opening of the positioned probe to draw at least a portion of the tissue to be ablated into contact with the at least one opening of the probe; and
applying electrical energy through the at least one electrode to electrically ablate the tissue.

22. The method of claim 20, further comprising applying electrical pulses to the probe in an amount which is sufficient to induce irreversible electroporation of cells of the target tissue, but which is insufficient to induce thermal damage to substantially all of the target tissue such that the identified tissue cells are killed by irreversible electroporation.

23. The method of claim 20, wherein the method further comprises before the step of applying suction, applying electrical energy to the electrodes to electrically ablate the target tissue.

24. The method of claim 20, wherein the target tissue comprises one or more of a cyst, cancerous tissue, uterine fibroids, a tumor with a necrotic core, a polyp, a lesion, a vessel, a duct, an aneurysm, and a body cavity.

25. The method of claim 23, wherein the method further comprises collapsing the aneurysm in or near the aneurysm during the step of applying electrical energy to the at least one electrode.

26. The method of claim 20, further comprising infusing at least one agent into the target tissue before, during, or after the step of applying suction, or any combination thereof, wherein the agent is selected from the group comprising: saline, D5W, an iso-tonic solution, a hypo-tonic solution, and a hyper-tonic solution.

27. The method of claim 20, wherein during the step of applying electrical energy, suction is maintained.

28. The method of claim 20, wherein the method further comprises stabilizing the target tissue in relationship to the probe during the step of applying suction to the target tissue.

29. The method of claim 20, wherein the probe comprises a lumen extending along a longitudinal axis of the probe, and wherein suction is applied through the lumen of the probe.

30. The method of claim 20, wherein the probe comprises a lumen, and wherein before, during, or after the step of applying electrical energy, or any combination thereof, at least one agent is infused into the lumen of the probe, wherein the agent is selected from the group comprising: saline, D5W, an iso-tonic solution, a hypo-tonic solution, and a hyper-tonic solution.

31. The method of claim 21, wherein the method further comprises removing at least a portion of the target tissue through suction, wherein the target tissue comprises a liquid.

32. A probe assembly comprising a probe having a proximal end, a distal end, an outer surface, and a tissue contacting surface, wherein the tissue contacting surface comprises a deployable structure, wherein at least a portion of the deployable structure surrounds at least a portion of the outer surface of the probe, wherein the deployable structure is configured to be fixed to the outer surface of the probe and the tissue.
